(19) United States
(12) Patent Application Publication
(10) Pub. No.: US 2017/0168812 A1
(43) Pub. Date: Jun. 15, 2017

(54) Integration of non-supported dental imaging devices into legacy and proprietary dental imaging software

(71) Applicants: Douglas A. Golay, Coon Rapids, IA (US); Wyatt C. Davis, Bozeman, MT (US)

(72) Inventors: Douglas A. Golay, Coon Rapids, IA (US); Wyatt C. Davis, Bozeman, MT (US)

(21) Appl. No.: 14/967,322

(22) Filed: Dec. 13, 2015

(51) Int. Cl. G06F 9/445; A61B 1/24; G06F 9/54; A61B 6/14

(52) U.S. Cl. CPC G06F 8/665; A61B 6/145; A61B 1/24; G06F 9/541

(57) ABSTRACT

A method for integrating a non-supported dental imaging device into dental imaging software operates on a computer which is coupled to a display that is capable of displaying dental x-rays and dental photographs. An originally supported dental imaging device has an API binary file with an original filename accessible to the computer. The method includes the steps of creating a replacement alternate API binary file which contains equivalent functionality as the API binary file of the original supported dental imaging device and placing the replacement alternate API binary file either onto or accessible to the computer. The replacement alternate API binary file has the same filename as does the original filename of the API binary file of the originally supported dental imaging device. The method also includes the step of having the replacement alternate API binary file operated on by the dental imaging software by means of the computer. The dental imaging software is not aware the dental imaging software is not communicating with the originally supported dental imaging device. The replacement alternate API binary file delivers image data acquired by the non-supported imaging device to the dental imaging software.

### BACKGROUND OF THE INVENTION

Field of the Invention

This present invention relates generally to dental imaging software and more particularly to integration of non-supported dental imaging devices into legacy and proprietary dental imaging software.

Description of the Prior Art

In the field of dentistry many vendors provide dental imaging software and imaging devices with associated hardware. Many of these vendors allow the user to mix and match various imaging devices from other manufacturers using a third party dental imaging software in order to allow other brands of imaging devices to operate within each vendor's own dental imaging software that is provided with its specific imaging devices thereby allowing dentists to acquire and store their images using a single dental imaging software regardless of either the imaging devices or the data imaging source in use by the dentist in his dental practice. Standards such as Dicom, Twain and others help facilitate this to some degree. Most imaging devices that are controlled by dental imaging software are directly integrated by means of proprietary device application programming interfaces (APIs) that allow maximum control of the sensors and other parameters during acquisition. These images may ultimately be “stored” in a Dicom format or on a Picture Archiving Communication System (PACS), but the acquisition is done by proprietary software and algorithms that are programmed for each imaging device or data image source that is supported by that specific dental imaging software.

Some dental imaging software companies intentionally do not support open standards, such as Dicom, and do not directly integrate with specific imaging devices for the sole reason that they offer a competitive imaging device. This is highly undesirable for the dentists as in this situation for them to mix and match imaging equipment brands they and their staff will have to operate more than one dental imaging software, some images will be in one imaging software and some will be in another dental imaging software. The added expense of buying, owning and training staff to use two separate dental imaging software is burdensome. PACS/DICOM are not used often in general dentistry offices because of added complexity of such servers, maintenance and costs. Dentists in their dental offices do not typically have information technology (IT) employees on staff to monitor and maintain these more complex systems. The two largest providers of 2D intraoral x-ray sensors (Schick/Sirona and DEXIS/Gendex) do not publish any open Application Programming Interfaces (APIs) for integration of imaging devices into their dental imaging software and physically do not add support to their imaging software for specific third party intraoral x-ray sensors that they and/or their distributors cannot sell and/or are competitive to their other brands. There are several legacy dental imaging software that are not typically updated often, if at all. In these cases it is burdensome for a dentist to have to change his dental imaging software so that he may not be able to either import or convert all of his images from legacy applications and will have to buy a new dental imaging software and retrain his staff on the new dental imaging software. It would be highly desirable for any current imaging devices to be supported in these legacy applications in order for a dentist to continue using the dental imaging software he owns and uses now that still allows the dentist to use any manufacturer's intraoral or extraoral x-ray sensor and an imaging device with any legacy or proprietary dental imaging software that does not support that specific imaging device directly using open standards.

Intraoral and extraoral x-rays have been used in dentistry to image teeth, jaw, and facial features for many years. In general this process involves generating x-rays from an intraoral or extraoral x-ray source, directing the x-ray source at the patient's oral cavity and placing an image receptor device, such as film or a digital sensor, to receive x-rays from either the intra-oral or extra-oral x-ray source. The x-rays generated by the x-ray source are attenuated by different amounts depending on the dental structure being x-rayed and whether it is bone or tissue. The resulting x-ray photons that pass thru the bone or tissue then form an image on the image receptor whether it is film or a form of an electronic/organic image sensor/detector. This image/data is then either developed in the case of film or processed and displayed on a computer monitor in the case of an imaging sensor. The intraoral and extraoral sensors are controlled by and deliver images to a dental imaging software operating on a computer that includes a microprocessor, a random access memory (RAM), a storage device, a bus, a display monitor and other physical hardware devices. When an intraoral or extraoral sensor is used in combination with the dental imaging software that has been optimized for dentistry a functional system is created that dentists and other dental caregivers can use to diagnose and treat patient dental conditions.

U.S. Patent Publication No. 2013/0226993 teaches a media acquisition engine which includes an interface engine that receives a selection from a plug-in coupled to a media client engine where a client associated with the media client engine identified as subscribing to a cloud application imaging service. The media acquisition engine also includes a media control engine that directs, in accordance with the selection, a physical device to image a physical object and produce a media item based on the image of the physical object, the physical device being coupled to a cloud client. The media acquisition engine also includes a media reception engine that receives the media item from the physical device and a translation engine that encodes the media item into a data structure compatible with the cloud application imaging service. The interface engine is configured to transfer the media item to the plug-in. Digital imaging has notable advantages over traditional imaging, which processes an image of a physical object onto a physical medium. Digital imaging help users such as health professionals avoid the costs of expensive processing equipment, physical paper, physical radiographs, and physical film. Techniques such as digital radiography expose patients to lower doses of radiation than traditional radiography and are often safer than their traditional counterparts are. Digital images are easy to store on storage such as a computer's hard drive or a flash memory card, are easily transferable and are more portable than traditional physical images. Many digital imaging devices use sophisticated image manipulation techniques and filters that accurately image physical objects. A health professional's information infrastructures and the business processes can therefore potentially benefit from digital imaging techniques. Though digital imaging has many advantages over physical imaging, digital imaging technologies are far from ubiquitous in health offices as existing digital imaging technologies present their own costs. To use existing digital imaging technologies, a user such as a health professional has to purchase separate computer terminals and software licenses for each treatment room. As existing technologies install a full digital imaging package on each computer terminal, these technologies are often expensive and present users with more options than they are willing to pay for. Additionally, existing digital imaging technologies require users to purchase a complete network infrastructure to support separate medical imaging terminals. Users often face the prospects of ensuring software installed at separate terminals maintains patient confidentiality, accurately stores and backs up data, accurately upgrades, and correctly performs maintenance tasks. Existing digital imaging technologies are not readily compatible with the objectives of end-users, such as health professionals.

Referring to FIG. 1 a networking system 100 includes a desktop computer 102, a laptop computer 104, a server 106, a network 108, a server 110 a server 112, a tablet device 114 and a private network group 120 in order to provide at least one or more application imaging services. The private network group 120 includes a laptop computer 122, a desktop computer 124, a scanner 126, a tablet device 128, an access gateway 132, a first physical device 134, a second physical device 136 and a third physical device 138. The desktop computer 102, the laptop computer 104, the server 106, the server 110 the server 112, and the tablet device 114 are directly connected to the network 108. The desktop computer 102 may include a computer having a separate keyboard, a mouse, a display/monitor and a microprocessor. The desktop computer 102 can integrate one or more of the keyboard, the monitor, and the processing unit into a common physical module. The laptop computer 104 can include a portable computer. The laptop 104 can integrate a keyboard, a mouse, a display/monitor and a microprocessor into one physical unit. The laptop 104 also has a battery so that the laptop 104 allows portable data processing and portable access to the network 108. The tablet 114 can include a portable device with a touch screen, a display/monitor, and a processing unit all integrated into one physical unit. Any or all of the computer 102, the laptop 104 and the tablet device 118 may include a computer system. A computer system will usually include a microprocessor, a memory, a non-volatile storage and an interface. Peripheral devices can also form a part of the computer system. A typical computer system will include at least a processor, memory, and a bus coupling the memory to the processor. The processor can include a general-purpose central processing unit (CPU), such as a microprocessor or a special-purpose processor, such as a microcontroller. The memory can include random access memory (RAM), such as dynamic RAM (DRAM) and static RAM (SRAM). The memory can be local, remote, or distributed. The term “computer-readable storage medium” includes physical media, such as memory. The bus of the computer system can couple the processor to non-volatile storage. The non-volatile storage is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. A direct memory access process often writes some of this data into memory during execution of software on the computer system. The non-volatile storage can be local, remote, or distributed. The non-volatile storage is optional because systems need only have all applicable data available in memory. Software is typically stored in the non-volatile storage. Indeed, for large programs, it may not even be possible to store the entire program in memory. Nevertheless, for software to run, if necessary, it is moved to a computer-readable location appropriate for processing. Even when software is moved to the memory for execution, the processor will make use of hardware registers to store values associated with the software, and local cache that, ideally, serves to speed up execution. A software program is assumed to be stored at any known or convenient location from non-volatile storage to hardware registers when the software program is referred to as “implemented in a computer-readable storage medium.” A microprocessor is “configured to execute a program” when at least one value associated with the program is stored in a register readable by the microprocessor. The bus can also couple the microprocessor to one or more interfaces. The interface can include one or more of a modem or network interface. A modem or network interface can be part of the computer system. The interface can include an analog modem, ISDN modem, cable modem, token ring interface, satellite transmission interface or other interfaces for coupling a computer system to other computer systems. The interface can include one or more input and/or output (I/O) devices. The I/O devices can include a keyboard, a mouse or other pointing device, disk drives, printers, a scanner, and other I/O devices, including a display/monitor. The display/monitor device can include a cathode ray tube (CRT), liquid crystal display (LCD) or some other applicable known or convenient display device. Operating system software includes a file management system, such as a disk operating system, can control the computer system. One operating system software with associated file management system software is the family of operating systems known as Windows from Microsoft Corporation of Redmond, Wash., and their associated file management systems. Another operating system software with its associated file management system software is the Linux operating system and its associated file management system. The file management system is typically stored in the non-volatile storage and causes the processor to execute the various acts required by the operating system to input and output data and to store data in the memory, including storing files on the non-volatile storage. Some portions of the detailed description refer to algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. All of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices. The algorithms and displays presented herein do not inherently relate to any particular computer or other apparatus. Any or all of the computer 102, the laptop 104 and the tablet device 118 can include engines. As used in this paper, an engine includes a dedicated or shared processor and, typically, firmware or software modules that the processor executes. Depending upon implementation-specific or other considerations, an engine can have a centralized distributed location and/or functionality. An engine can include special purpose hardware, firmware, or software embodied in a computer-readable medium for execution by the processor. A computer-readable medium is intended to include all mediums that are statutory (e.g., in the United States, under 35 U.S.C. 101), and to specifically exclude all mediums that are non-statutory in nature to the extent that the exclusion is necessary for a claim that includes the computer-readable medium to be valid. Known statutory computer-readable mediums include hardware (e.g., registers, random access memory (RAM), non-volatile (NV) storage, to name a few), but may or may not be limited to hardware. Any or all of the computer 102, the laptop 104 and the tablet device 118 can include one or more data-stores. A data-store can be implemented as software embodied in a physical computer-readable medium on a general- or specific-purpose machine, in firmware, in hardware and in a combination thereof, or in an applicable known or convenient device or system. Data-stores in this paper are intended to include any organization of data, including tables, comma-separated values (CSV) files, traditional databases (e.g., SQL), or other applicable known or convenient organizational formats. Data-store-associated components, such as database interfaces, can be considered “part of” a data-store, part of some other system component, or a combination thereof, though the physical location and other characteristics of data-store-associated components is not critical for an understanding of the techniques described in this paper. Data-stores can include data structures. A data structure is associated with a particular way of storing and organizing data in a computer so for efficient use within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The desktop computer 102, the laptop 104 or the tablet device 114 can function as network clients. Any or all of the desktop computer 102, the laptop 104 and the tablet device 114 can include one or more operating system software as well as application system software. The desktop computer 102, the laptop 104 or the tablet device 114 run a version of a Windows operating system from Microsoft Corporation, a version of a Mac operating system from Apple Corporation, a Linux based operating system such as an Android operating system, a Symbian operating system, a Blackberry operating system or other operating system. The desktop computer 102, the laptop 104 and the tablet device 114 can also run one or more applications with which end-users can interact. The desktop computer 102, the laptop 104 and the tablet device 114 can run word processing applications, spreadsheet applications, imaging applications and other applications. Any or all of the desktop computer 102, the laptop 104 and the tablet device 114 can also run one or more programs that allow a user to access content over the network 108. Any or all of the desktop computer 102, the laptop 104 and the tablet device 114 can include one or more web browsers that access information over the network 108 by Hypertext Transfer Protocol (HTTP). The desktop computer 102, the laptop 104 and the tablet device 114 can also include applications that access content via File Transfer Protocols (FTP) or other standards. The desktop computer 102, the laptop 104 or the tablet device 114 can also function as servers. A server is an electronic device that includes one or more engines dedicated in whole or in part to serving the needs or requests of other programs and/or devices. The discussion of the servers 106, 110 and 112 provides further details of servers.

Referring to FIG. 2 in conjunction with FIG. 1 the desktop computer 102, the laptop 104 or the tablet device 114 can distribute data and/or processing functionality across the network 108 to facilitate providing cloud application imaging services. Any of the desktop computer 102, the laptop 104 and the tablet device 114 can incorporate modules such as the cloud-based server engine 200. Any of the server 106, the server 110 and the server 112 can include computer systems. Any of the server 106, the server 110 and the server 112 can include one or more engines. Any of the server 106, the server 110 and the server 112 can incorporate one or more data-stores. The engines in any of the server 106, the server 110 and the server 112 can be are dedicated in whole or in part to serving the needs or requests of other programs and/or devices. Any of the server 106, the server 110 and the server 112 can handle relatively high processing and/or memory volumes and relatively fast network connections and/or throughput. The server 106, the server 110 and the server 112 may or may not have device interfaces and/or graphical user interfaces (GUIs). Any of the server 106, the server 110 and the server 112 can meet or exceed high availability standards. The server 106, the server 110 and the server 112 can incorporate robust hardware, hardware redundancy, network clustering technology, or load balancing technologies to ensure availability. The server 106, the server 110 and the server 112 can incorporate administration engines that from electronic devices such as the desktop computer 102, the laptop computer 104 or the tablet device 114, or other devices access remotely through the network 108. Any of the server 106, the server 110 and the server 112 can include an operating system that is configured for server functionality, i.e., to provide services relating to the needs or requests of other programs and/or devices. The operating system in the server 106, the server 110 or the server 112 can include advanced or distributed backup capabilities, advanced or distributed automation modules and/or engines, disaster recovery modules, transparent transfer of information and/or data between various internal storage devices as well as across the network, and advanced system security with the ability to encrypt and protect information regarding data, items stored in memory, and resources. The server 106, the server 110 and the server 112 can incorporate a version of a Windows server operating system from Microsoft Corporation, a version of a Mac server operating system from Apple Corporation, a Linux based server operating system, a UNIX based server operating system, a Symbian server operating system, a Blackberry server operating system, or other operating system. The server 106, the server 110 and the server 112 can distribute functionality and/or data storage. The server 106, the server 110 and the server 112 can distribute the functionality of an application server and can therefore run different portions of one or more applications concurrently. Each of the server 106, the server 110 and the server 112 stores and/or executes distributed portions of application services, communication services, database services, web and/or network services, storage services, and/or other services. The server 106, the server 110 and the server 112 can distribute storage of different engines or portions of engines. For instance, any of the server 106, the server 110 and the server 112 can include some or all of the engines shown in the cloud-based server engine 200. The networking system 100 can include the network 108. The network 108 can include a networked system that includes several computer systems coupled, such as a local area network (LAN), the Internet, or some other networked system. The term “Internet” as used in this paper refers to a network of networks that uses certain protocols, such as the TCP/IP protocol, and possibly other protocols such as the HTTP for hypertext markup language (HTML) documents that make up the World Wide Web. Content servers, which are “on” the Internet, often provide the content. A web server, which is one type of content server, is typically at least one computer system, which operates as a server computer system, operates with the protocols of the World Wide Web, and has a connection to the Internet. Applicable known or convenient physical connections of the Internet and the protocols and communication procedures of the Internet and the web are and/or can be used. The network 108 can broadly include anything from a minimalist coupling of the components illustrated to every component of the Internet and networks coupled to the Internet. Components that are outside of the control of the networking system 100 are sources of data received in an applicable known or convenient manner. The network 108 can use wired or wireless technologies, alone or in combination, to connect the devices inside the networking system 100. Wired technologies connect devices using a physical cable such as an Ethernet cable, digital signal link lines (T1-T3 lines), or other network cable. The private network group 120 includes a wired local area network wired personal area network (PAN), a wired LAN, a wired metropolitan area network, or a wired wide area network. Some or all of the network 108 may include cables that facilitate transmission of electrical, optical, or other wired signals. Some or all of the network 108 can also employ wireless network technologies that use electromagnetic waves at frequencies such as radio frequencies (RF) or microwave frequencies. The network 108 includes transmitters, receivers, base stations, and other equipment that facilitates communication via electromagnetic waves. Some or all of the network 108 may include a wireless personal area network (WPAN) technology, a wireless local area network (WLAN) technology, a wireless metropolitan area network technology, or a wireless wide area network technology. The network 108 can use Global System for Mobile Communications (GSM) technologies, personal communications service (PCS) technologies, third generation (3G) wireless network technologies, or fourth generation (4G) network technologies. The network 108 may also include all or portions of a Wireless Fidelity (Wi-Fi) network, a Worldwide Interoperability for Microwave Access (WiMAX) network, or other wireless network. The networking system 100 can include the private network group 120. The private network group 120 is a group of computers that form a subset of the larger network 108. The private network group 120 can include the laptop computer 122, the desktop computer 124, the scanner 126, the tablet device 128, the access gateway 132, the first physical device 134, the second physical device 136 and the third physical device 138. The laptop computer 122 can be similar to the laptop computer 104 the desktop computer 124 can be similar to the desktop computer 102, and the tablet device 128 can be similar to the tablet device 114. Any of the laptop computer 122, the desktop computer 124, the scanner 126, the tablet device 128, the access gateway 132, the first physical device 134, the second physical device 136 and the third physical device 138 can include computer systems, engines, data-stores. The private network group 120 can include a private network. A private network provides a set of private internet protocol (IP) addresses to each of its members while maintaining a connection to a larger network, here the network 108. To this end, the members of the private network group 120 (i.e., the laptop computer 122, the desktop computer 124, the scanner 126, the tablet device 128, the first physical device 134, the second physical device 136 and the third physical device 138) can each be assigned a private IP address irrespective of the public IP address of the router 132. Though the term “private” appears in conjunction with the name of the private network group 120 the private network group 120 includes a public network that forms a subset of the network 108. In such a case, each of the laptop computer 122, the desktop computer 124, the scanner 126, the tablet device 128, the first physical device 134, the second physical device 136 and the third physical device 138 can have a public IP address and can maintain a connection to the network 120. The connection of some or all of the laptop computer 122, the desktop computer 124, the scanner 126, the tablet device 128, the first physical device 134, the second physical device 136 and the third physical device 138 can be a wired or a wireless connection. The private network group 120 includes the access gateway 132. The access gateway 132 assigns private IP addresses to each of the devices 122, 124, 126, 128, 134, 136 and 138. The access gateway 132 can establish user accounts for each of the devices 122, 124, 126, 128, 134, 136 and 138 and can restrict access to the network 108 based on parameters of those user accounts. The access gateway 132 can also function as an intermediary to provide content from the network 108 to the devices 122, 124, 126, 128, 134, 136 and 138. The access gateway 132 can format and appropriately forward data packets traveling over the network 108 to and from the devices 122, 124, 126, 128, 134, 136 and 138. The access gateway 132 can be a router, a bridge, or other access device. The access gateway 132 can maintain a firewall to control communications coming into the private network group 120 through the network 108. The access gateway 132 can also control public IP addresses associated with each of the laptop computer 122, the desktop computer 124, the scanner 126, the tablet device 128, the first physical device 134, the second physical device 136 and the third physical device 138. The access gateway 132 is absent and each of the devices inside the private network group 120 can maintain its own connection to the network 108. The desktop computer 124 is shown connected to the access gateway 132 as such a configuration is a common implementation. The functions described in relation to the desktop computer 124 can be implemented on the laptop computer 122, the tablet device 128, or any applicable computing device. The private network group 120 can be located inside a common geographical area or region. The private network group 120 can be located in a school, a residence, a business, a campus, or other location. The private network group 120 is located inside a health office, such as the office of a dentist, a doctor, a chiropractor, a psychologist, a veterinarian, a dietician, a wellness specialist, or other health professional. The physical devices 134, 136 and 138 can image a physical object. The physical devices 134, 136 and 138 can connect to the desktop computer 124 via a network connection or an output port of the desktop computer 124. Similarly, the physical devices 134, 136 and 138 can connect to the laptop computer 122, the tablet device 128, or a mobile phone. The physical devices 134, 136 and 138 are directly connected to the access gateway 132. The physical devices 134, 136 and 138 can also internally incorporate network adapters that allow a direct connection to the network 108. The first physical device 134 can be a sensor-based imaging technology. A sensor is a device with electronic, mechanical, or other components that measures a quantity from the physical world and translates the quantity into a data structure or signal that a computer, machine, or other instrument can read. The first physical device 134 can use a sensor to sense an attribute of a physical object. The physical object can include, for instance, portions of a person's mouth, head, neck, limb, or other body part. The physical object can be an animate or inanimate item. The sensor may include x-ray sensors to determine the boundaries of uniformly or non-uniformly composed material such as part of the human body. The sensor can be part of a Flat Panel Detector (FPD). Such an FPD can be an indirect FPD including amorphous silicon or other similar material used along with a scintillator. The indirect FPD can allow the conversion of X-ray energy to light, which is eventually translated into a digital signal. Thin Film Transistors (TFTs) or Charge Coupled Devices (CCDs) can subsequently allow imaging of the converted signal. Such an FPD can also be a direct FPD that uses Amorphous Selenium or other similar material. The direct FPD can allow for the direct conversion of x-ray photons to charge patterns that, in turn, are converted to images by an array such as a TFT array, an Active Matrix Array, or by Electrometer Probes and/or Micro-plasma Line Addressing. The sensor may also include a High Density Line Scan Solid State detector. The sensor of the first physical device 134 may include an oral sensor. An oral sensor is a sensor that a user such as a health practitioner can insert into a patient's mouth. The first physical device 134 can reside in a dentist's office that operates the private network group 120. The sensor of the first physical device 134 may also include a sensor that is inserted into a person's ear, nose, throat or other part of a person's body. The second physical device 136 may include a digital radiography device. Radiography uses x-rays to view the boundaries of uniformly or non-uniformly composed material such as part of the human body. Digital radiography is the performance of radiography without the requirements of chemical processing or physical media. Digital radiography allows for the easy conversion of an image to a digital format. The digital radiography device can be located in the office of a health professional. The third physical device 138 may include a thermal-based imaging technology. Thermal imaging technology is technology that detects the presence of radiation the infrared ranges of the electromagnetic spectrum. Thermal imaging technology allows the imaging of the amount of thermal radiation emitted by an object. The third physical device 138 may include an oral sensor, or a sensor that is inserted into a person's ear, nose, throat, or other part of a person's body. The third physical device 138 resides in the office of a health professional, such as the office of a dentist, a doctor, a chiropractor, a psychologist, a veterinarian, a dietician, a wellness specialist or other health professional. The networking system 100 can facilitate delivery of a cloud application imaging service. A cloud application imaging service is a service that allows an entity associated with a physical device (such as one of the physical devices 134, 136 and 138) to use a cloud-computing application that is executed on a client computer (such as the desktop computer 124) to direct the physical device to image a physical object. Cloud-based computing, or cloud computing, is a computing architecture in which a client can execute the full capabilities of an application in a container (such as a web browser). Though the application executes on the client, portions of the application can be distributed at various locations across the network. Portions of the cloud application imaging service that are facilitated by the networking system 100 can reside on one or more of the desktop computer 102, the laptop computer 104, the server 106, the server 110 the server 112, the tablet device 114, and/or other locations “in the cloud” of the networking system 100. The application can appear as a single point of access for an end-user using a client device such as the desktop computer 124. The cloud application imaging service can implement cloud client functionalities onto the desktop computer 124. A cloud client incorporates hardware and/or software that allows a cloud application to run in a container such as a web browser. Allowing the desktop computer 124 to function as a cloud client requires the presence of a container in which the cloud application imaging service can execute on the desktop computer 124. The cloud application imaging service can facilitate communication over a cloud application layer between the client engines on the desktop computer 124 and the one or more server engines on the desktop computer 102, the laptop computer 104, the server 106, the server 110 the server 112, the tablet device 114, and/or other locations “in the cloud” of the networking system 100. The cloud application layer or “Software as a Service” (SaaS) facilitates the transfer over the Internet of software as a service that a container, such as a web browser, can access. Thus, as discussed above, the desktop computer 124 need not install the cloud application imaging service even though the cloud application imaging service executes on the desktop computer 124. The cloud application imaging service can also deliver to the desktop computer 124 one or more Cloud Platform as a Service (PaaS) platforms that provide computing platforms, solution stacks, and other similar hardware and software platforms. The cloud application imaging service can deliver cloud infrastructure services, such as Infrastructure as a Service (IaaS) that can virtualize and/or emulate various platforms, provide storage, and provide networking capabilities. The cloud application imaging service, consistent with cloud-computing services in general, allows users of the desktop computer 124 to subscribe to specific resources that are desirable for imaging and other tasks related to the physical devices 134, 136 and 138. Providers of the cloud application imaging service can bill end-users on a utility computing basis, and can bill for use of resources. In the health context, providers of the cloud application imaging service can bill for items such as the number of images an office wishes to process, specific image filters that an office wishes to use, and other use-related factors.

Referring to FIG. 2 either part or all of the cloud application imaging service can reside on one or more server engines. A conceptual diagram of a cloud-based server engine 200 includes a device search engine 202 that searches the physical devices connected to a client computer. The cloud-based server engine 200 may also include remote storage 204 that includes one or more data-stores and/or memory units. The remote storage 204 can include storage on Apache-based servers that are available on a cloud platform such as the EC2 cloud platform made available by Amazon. The cloud-based server engine 200 can may include a physical device selection engine 206 that selects a specific physical device connected to a client. The cloud-based server engine 200 can include a physical device configuration engine 208 that configures image parameters and/or attributes of the specific physical device. An image selection engine 210 inside the cloud-based server engine 200 can allow the selection of a specific image from the physical device. A communication engine 212 inside the cloud-based server engine 200 allow the transfer of selection data, parameter data, device data, image data, and other data over a network such as the network 108. The cloud-based server engine 200 includes a content engine 214 that makes images available to client devices associated with a cloud application imaging service. Processors can control any or all of the components of the cloud-based server engine 200 and these components can interface with data-stores. Any or all of the cloud-based server engine 200 can reside on a computing device such as the desktop computer 102, the laptop 104, the tablet device 114, the server 106, the server 110 and/or server 112. Portions of the cloud-based server engine 200 can also be distributed across multiple electronic devices, including multiple servers and computers.

Referring to FIG. 3 in conjunction with FIG. 1 and FIG. 2 a cloud-based client system 300 includes the network 108, the first physical device 134, the second physical device 136 and the third physical device 138. Each of the network 108, the first physical device 134, the second physical device 136 and the third physical device 138. The cloud-based client system 300 also includes a cloud-based media acquisition client 304 which can reside inside a computer, such as the desktop computer 124. The cloud-based media acquisition client 304 also interfaces with the network 108. The access gateway 132 allows the cloud-based media acquisition client 304 to communicate with the network 108. The cloud-based media acquisition client 304 can also be connected to the network 108 through other I/O devices and/or means. The cloud-based media acquisition client 304 is also connected to the first physical device 134, the second physical device 136 and the third physical device 138. Either a network connection or an I/O device and/or means can facilitate the connections between the cloud-based media acquisition client 304 and any of the first physical device 134, the second physical device 136 and the third physical device 138.

U.S. Pat. No. 5,434,418 teaches an intra-oral sensor for computer aided oral examination by means of low dosage x-rays in place of film and developer. A signal thereafter causes a read out of the electrical charges for translation from analog to digital signals of images with computer display and analysis. Dentists and oral surgeons typically utilize x-ray apparatus to examine patients prior to treatment. Film placed in the patient's mouth is exposed to the x-rays which pass through the soft tissue of skin and gums and are absorbed or refracted by the harder bone and teeth structures. The film is then chemically developed and dried to produce the image from which the dentist makes appropriate treatment; evaluations. Such technology, though with many refinements, has not basically changed over the past fifty years. Though the technology is a mature one and well understood in the field, there are numerous drawbacks in conventional dental radiology which utilizes film for image capturing. Foremost among such problems is the radiation dosage, which optimally for conventional film exposure, is about 260 milliradians. Since the high energy electrons from x-ray sources can cause damage to the nuclei of cells minimizing radiation exposure is highly desirable. The average dose for dental x-rays has been reduced by 50% over the last thirty years, to the current levels, mostly as a result of improvement in film sensitivity. Further incremental reductions in requisite x-ray dosage for film exposure is unlikely to be of any great extent. Film processing itself presents other problems including the time, expense, inconvenience and uncertainty of processing x-ray films and many times the exposure is defective or blurred. The minimum time for development is four to six minutes. There is the cost and inconvenience of storing and disposing of the developing chemicals which are usually environmentally harmful. The additional components entail greater costs, introduce problems with component degradation and failure, and generally preclude direct sterilization by dental autoclaving. The intraoral sensor is connected to a small radio transmitter for image transmission to a remote computer. In operation the intra-oral sensor translates the x-rays to light which then generates an analog signal. The analog signal then causes a read out of the electrical charges for translation from analog to digital signals of images with computer display and analysis. The sensor is attached via the thin, flexible PTFE cable to an interface box, which is connected to the computer. The interface box digitizes the analog video signal from the sensor and transmits it to the computer for the display and analysis. The computer, and associated peripherals, used to acquire the images from the sensor incorporates a CPU, a removable disk sub-system, a high-resolution display system, a printer which can reproduce an entire image with at least 256 shades of grey, a keypad and a mouse/pointing device. The CPU has sufficient power to execute digital signal processing (DSP) routines on the images without noticeable time-lag. The removable disk sub-system stores the images. The high-resolution display system displays colors and at least 256 shades of gray. The printer reproduces an entire image with at least 256 shades of grey. The keypad and the mouse/pointing device act as an operator interface. Optional devices for additional enhancements include a high-speed modem to transmit x-ray image data in order to take full advantage of automatic insurance claims processing, a write once optical-disk subsystem for mass storage of images and a local-area network to connect more than one system within an office. Software for operation of the system includes software which allows the dentist an easy and intuitive method of taking x-rays, organizing and viewing them, and storing and recalling them. On a low-level, the software controls the sensor operation and other system functions. The software also includes a set of algorithms for manipulating the images via image compression routines with variable compression rate and image quality, filter routines for noise elimination and feature enhancement, contrast equalization, expansion and adjustment algorithms and viewing routines for zooming and panning. The normal exposure sequence is conducted as follows. The dentist positions the sensor in the patient's mouth and sets the computer to begin monitoring. The computer holds the array in a reset mode which clears all of the pixels and begins polling the discrete photodiodes. As soon as the exposure begins, the computer senses current across the diodes. The array is placed in an exposure mode in which the pixels are allowed to integrate the accumulated charge. When the exposure ends the computer senses that the diodes are not conducting. A clock signal is applied to the array to read out the image.

Referring to FIG. 4 an analog signal from a sensor 401 enters an interface box 417 which digitizes the signal for computer processing by a CPU unit 411. The digitized signal is thereafter directly carried by the cable 402 to the CPU unit 411 a short (14″ or 36 cm) cable 402 to a short range radio transmitter 420 (with internal analog to digital converter) for transmission to a receiver 421 and then to the CPU unit 411. The sensor 401 and the attached cable 402 are auto-claviable with the cable 402 being detachable from the interface box 417 and the radio transmitter 420. The processing can be made available on a network 416 or to a single output device such as either a monitor 412 or a printer 414. Appropriate instructions and manipulation of image data is effected via a keyboard 415 or input control. The x-ray images can thereafter be efficiently directly transmitted to remote insurance carrier computers via an internal modem and standard telephone line.

U.S. Pat. No. 6,091,982 teaches a mobile signal pick-up means of a diagnostics installation includes a radiation receiver for generating electrical signals dependent on the radiation shadow of a trans-irradiated subject, an image acquisition system, a calculating and storage unit, a display as well as a communication means. A stationary evaluation means includes a communication means which is implemented as bidirectional communication means and serves for the signal transmission between the mobile signal pick-up means and the stationary evaluation means. A medical diagnostic installation of the type has a mobile signal pick-up unit in communication with at least one stationary evaluation unit. The stationary evaluation unit is disposed remote from the mobile signal pick-up unit. The x-ray diagnostic installations are known that include a pick-up unit composed of a radiation transmitter and a radiation receiver as well as a stationary evaluation unit in communication therewith. The electrical signals acquired upon trans-irradiation of a subject are thereby supplied to the stationary evaluation unit via cable. The stationary evaluation unit converts these signals into image signals that can then be displayed on a monitor as an image of the subject. An x-ray image acquisition card can be provided with suitable means for wireless transmission of the data from a sensor into the computer unit (lap top), whereby the means for the wireless transmission can be implemented as infrared transmission and reception means. An image of the examination subject can be displayed on the monitor of the computer unit (lap top).

Referring to FIG. 5 an x-ray diagnostics installation includes different rooms 501, 502, 503, 504 in which personal computers 505, 506, 507, 508 are provided as stationary evaluation units, these being in communication with one another via a data network 509. A mobile signal pick-up unit 511 is present in one room 502. This mobile signal pick-up unit 511 has a radiation receiver 512 that converts the radiation shadow produced upon trans-irradiation of an examination subject by radiation of a radiation transmitter 513 into electrical signals. The mobile signal pick-up unit 511 is in bi-directional communication with the network 509 and the computers 505, 506, 507, 508 via a base station 514.

Referring to FIG. 6 in conjunction with FIG. 5 the mobile signal pick-up unit 511 has a radiation receiver 512 whose signals are supplied to an image acquisition sub-system 515 that includes an analog stage 516, a radiation recognition stage 517, a local image memory 518 as well as a DMA stage 519. In the analog stage 516, the analog signals output from the radiation receiver 512 are converted into digital signals with an analog-to-digital converter. These digital signals are deposited in a random access memory (RAM), which can ensue especially fast when a sub-system (DMA) that has direct access to this memory is employed therefor. The image acquisition system is connected via a bus 20 to a local computer 521, a display 522, a network card 523 as well as further auxiliary cards 524. The radiation receiver 512 is operated in three phases: readiness (arbitrarily long); incident radiation is detected. After radiation detection has ensued, a fastest possible switch is made into the integration phase in which the radiation receiver 512 converts the x-ray shadow-gram into a two-dimensional charge image. Clocking the charge image out into the image acquisition sub-system 517 with subsequent digitization and transmission to the computer 505, 506, 507, 508. A replaceable accumulator 525 that can be supplied with energy from a stationary charging station 526 can be provided for voltage supply of the mobile signal pick-up unit 511. The charging station can thereby serve as stationary table or wall mount that enables an un-problematical and fast manipulation. So that the mobile signal pick-up unit 511 can be dimensioned small and cost-beneficially manufactured and exhibits a low power consumption, it is advantageous when no possibility for image display or for patient dialogue is provided. The user dialogue ensues via the computer or computers 505, 506, 507, 508. The mobile signal pick-up unit 511 displays status and/or error messages with an alphanumerical display or via LEDs. An LCD image display with a flat picture screen is also meaningful.

Referring to FIG. 7 in conjunction with FIG. 5 and FIG. 6 a network programming interface between the mobile signal pick-up unit 511 and the computer 505, 506, 507, 508. The mobile signal pick-up unit 511 includes a first block 627 for image generation, image transfer and communication between the mobile signal pick-up unit 511 and the computer or computers 505, 506, 507, 508 takes place. The image generation includes the three operating phases: readiness, radiation detection and clocking the signals of the radiation receiver 512 out via the analog-to-digital converter and the DMA and clocking them into the RAM of the mobile signal pick-up unit 511. The image transfer with transmission of the signals corresponding to the received radiation shadow ensues from the RAM to the computer or computers 505, 506, 507, 508 via a network card. The communication between the mobile signal pick-up unit 511 and the computer or computers 505, 506, 507, 508 thereby via a bidirectional transmission of not only image data but also for monitoring purposes, error messages and status displays. A further block 628 with respect to the network API (Application Programming Interface), a block 629 with respect to the operating system and a block 630 with respect to the network level are also provided. The block circuit diagram representing each of the computer or computers 505, 506, 507, 508 also contains blocks corresponding to the blocks 628 through 630, for example a server and a block 634 for the image processing of the image signals of the radiation receiver 512 for the patient selection and patient allocation as well as for image archiving. A plurality of steps that are passed in what are referred to as layers are required for the data transmission between the computer or computers 505, 506, 507, 508 as well as between the computer of the mobile signal pick-up unit 511 and the computer or computers 505, 506, 507, 508. These layers are partly standardized and appropriate software protocols exist therefor. This software must be present both on the mobile signal pick-up unit 511 as well as on the computer or computers 505, 506, 507, 508 so that the information to be transmitted (image data, status) can be exchanged between the blocks 627 and 634. A distributed client-server solution has thereby proven especially advantageous as software structure. As a result, an arbitrary plurality of mobile signal pick-up unit 511 can communicate via the network with what is likewise an arbitrary plurality of stationary evaluation means. The job of the client is the acquisition of the raw image data and the forwarding of these data to one of the computer or computers 505, 506, 507, 508. The latter has the job of further-processing these raw data and archiving them in patient-related fashion. The radiation receiver 512 can be not only for the conversion of an x-ray shadow-gram but can also be a means for measuring a 3-D image for tooth restoration (CEREC), an intro-oral color video camera for diagnosis, a means for measuring dental pocket depth, a means for measuring the tooth stability in the jaw (PERIOTEST), a means for measuring and checking the occlusion and/or a means for measuring chemical data (pH value) of the saliva. It will be apparent to those of ordinary skill in the art that each of the aforementioned means will include an appropriate control and acquisition system. It should be assured in the bidirectional communication that the signals are reliably transmitted from the mobile signal pick-up unit 511 to the computer or computers 505, 506, 507, 508 and in a form that the correct reception of the image data is acknowledged and potentially repeated in case of error.

U.S. Patent Publication No. 2011/0304740 teaches a universal image capture manager (UICM) which facilitates the acquisition of image data from a plurality of image source devices (ISDs) to an image utilizing software (IUSA). The universal image capture manager is implemented on a computer processing device and includes a first software communication interface configured to facilitate data communication between the universal image capture manager and the image utilizing software. The universal image capture manager also includes a translator/mapper (T/M) software component being in operative communication with the first software communication interface and configured to translate and map an image request from the image utilizing software to at least one device driver software component of a plurality of device driver software components. The universal image capture manager further includes a plurality of device driver software components being in operative communication with the translator/mapper software component. Each device driver software components is configured to facilitate data communication with at least one image source device. Many times it is desirable to bring images into a user software. This is often done in the context of a medical office environment or a hospital environment. Images may be captured by image source devices such as a digital camera device or an x-ray imaging device and are brought into a user software such as an imaging software or a practice management software running on either a personal computer or a workstation. Each image source device may require a different interface and image data format for acquiring image data from that image source device. The various interfaces may be TWAIN-compatible or not, may be in the form of an application program interface (API), a dynamic link library (DLL) or some other type of interface. The various image data may be raw image data, DICOM image data, 16-bit or 32-bit or 64-bit image data, or some other type of image data. The process of acquiring an image into a user software can be difficult and cumbersome. In order to acquire and place an image in a user software a user may have to first leave the software, open a hardware driver, set the device options, acquire the image, save the image to a local storage area, close the hardware driver, return to the software, locate the saved image, and read the image file from the local storage area. Hardware and software developers have developed proprietary interfaces to help solve this problem. Having a large number of proprietary interfaces has resulted in software developers having to write a driver for each different device to be supported. This has also resulted in hardware device manufacturers having to write a different driver for each software. General interoperability between user software and image source devices has been almost non-existent. The imaging modality may be an intra-oral x-ray modality, a pan-oral x-ray modality, an intra-oral visible light camera modality, or any other type of imaging modality associated with the system. The anatomy may be one or more teeth numbers, a full skull, or any other type of anatomy associated with the system. The operatory may be operatory #1, or operatory #4, a pan-oral operatory, an ultrasound operatory, or any other type of operatory associated with the system. The work-list may be a work0-list from a Picture Archiving and Communication System (PACS) server where the work-list includes a patient name. The specific hardware type may be a particular type of intra-oral sensor or a particular type of intra-oral camera. The patient type may be pediatric, geriatric, or adolescent. The interface is configured to access the clipboard of a computer processing device and paste the returned image data set to the clipboard. The universal image capture manager may be configured to enable all of the plurality of device drivers upon receipt of an image request message and, if any image source device of the plurality of image source devices has newly acquired image data to return, the newly acquired image data will be automatically returned to the image utilizing software through the universal image capture manager.

Referring to FIG. 8 a system 800 includes an image utilizing software (IUSA) 810 which is implemented on a first computer processing device 811, a universal image capture manager (universal image capture manager) 820 which is implemented on a second computer processing device 821 and a plurality of image source devices (ISDs) 830 (e.g., ISD #1 to ISD #N, where N represents a positive integer) in order to acquire image data from multiple sources. The image utilizing software 810 may be a client software such as an imaging software or a practice management application as may be used in a physician's office, a dentist's office, or a hospital environment. The image utilizing software 810 is implemented on the first computer processing device 811, such as a personal computer (PC) or a work station computer. There is a plurality of image source devices 830 which are hardware-based devices that are capable of capturing images in the form of image data (e.g., digital image data). Such image source devices 830 include a visible light intra-oral camera, an intraoral x-ray sensor, a panoramic (pan) x-ray machine, a cephalometric x-ray machine, a scanner for scanning photosensitive imaging plates and a digital endoscope. There exist many types of image source devices using many different types of interfaces and protocols to export the image data from the image source devices. The universal image capture manager 820 is a software or a software module. The second computer processing device 821, having the universal image capture manager 820, operatively interfaces between the first computer processing device 811, having the image utilizing software 810 and the plurality of image source devices 830, and acts as an intermediary between the image utilizing software 810 and the plurality of image source devices 830. The universal image capture manager 820 is a software module implemented on the second computer processing device 821 such as a personal computer, a workstation computer, a server computer, or a dedicated processing device designed specifically for universal image capture manager operation. The universal image capture manager 820 is configured to communicate in a single predefined manner with the image utilizing software 810 to receive image request messages from the image utilizing software image utilizing software 810 and to return image data to the image utilizing software 810. The universal image capture manager 820 is configured to acquire image data from the multiple image source devices 830. As a result, the image utilizing software 810 does not have to be concerned with being able to directly acquire image data from multiple different image data sources itself. Instead, the universal image capture manager 820 takes on the burden of communicating with the various image source devices 830 with their various communication interfaces and protocols.

Referring to FIG. 9 a universal image capture manager 820 (UICM) software module architecture used in the system 800 includes a first software interface that is a universal image capture manager/image utilizing software interface 910 that is configured to facilitate data communication between the universal image capture manager 820 and the image utilizing software 810. The interface 910 may be a USB interface, an Ethernet interface, or a proprietary direct connect interface. The interface 910 is implemented in software and operates with the hardware of the second computer processing device 821 to input and output data (e.g., image request message data and image data) from/to the image utilizing software 810. The universal image capture manager 820 further includes a plurality of device drivers 930 (e.g., DD #1 to device driver DD #N, where N is a positive integer). The device drivers 930 are implemented as software components and operate with the hardware of the second computer processing device 821 to input and output data (e.g., image data and device driver access data) from/to the plurality of image source devices 830. Each device driver 930 is configured to facilitate data communication with at least one of the image source devices 830. A device driver of the plurality of device drivers 930 may be a TWAIN-compatible device driver provided by a manufacturer of at least one corresponding image source device 830. TWAIN is a well-known standard software protocol that regulates communication between software and image source devices 830. TWAIN is not an official acronym but is widely known as “Technology without an Interesting Name.” Another device driver 930 may be a TWAIN-compatible or a non-TWAIN-compatible direct driver interface developed using a software development kit (SDK) provided by a manufacturer of at least one corresponding image source device 830. The software development kit includes a compiler, libraries, documentation, example code, an integrated development environment and a simulator for testing code. A device driver 930 may be a custom application programming interface (API). The application programming interface is an interface implemented by a software program which enables interaction with other software program. A device driver 930 may be part of a dynamic link library (DLL). The dynamic link library is a library that contains code and data that may be used by more than one software program at the same time and promotes code reuse and efficient memory usage. The universal image capture manager 820 is configured to be able to readily either add or remove a device driver software component to the plurality of device drivers 930. The universal image capture manager 820 is configured in a software “plug-n-play” manner so that device drivers may be readily either add device drivers or remove them without having to reconfigure any of the other device drivers. The universal image capture manager 820 may be easily adapted as image source devices 830 of the system 800 are able to add device drivers, change them out, upgraded them, replace them or discard them. The universal image capture manager 820 also includes a translator/mapper (T/M) 920 software component. The translator/mapper 920 is in operative communication with the universal image capture manager/image utilizing software interface 910 and the plurality of device drivers 930. The translator/mapper 920 is configured to translate and map an image request from the image utilizing software 810 to at least one device driver of the plurality of device drivers 930. The translator/mapper 920 is configured to translate and map image data received from at least one image source device of the plurality of image source devices 130 via at least one device driver of the plurality of device drivers 930. The computer-executable software instructions of the universal image capture manager 820 may be stored on a non-transitory computer-readable medium. The non-transitory computer-readable medium may include a compact disk (CDROM), a digital versatile disk (DVD), a hard drive, a flash memory, an optical disk drive, random access memory (RAM), read only memory (ROM), electronically erasable programmable read only memory (EEPROM), magnetic storage devices such as magnetic tape or magnetic disk storage, or any other medium that can be used to encode information that may be accessed by a computer processing device.

U.S. Patent Publication No. 2014/0350379 teaches a system for imaging a patient's body part that includes a non-transitory storage media to image the patient's body part. U.S. Patent Publication No. 2014/0350379 also teaches a method for imaging a patient's body part which includes the step of selecting an optical imaging device to image the patient's body part, the step of acquiring one or more data sets with the optical imaging device and the step of the acquiring is performed with focus at multiple axial positions and exposure control or deliberate focus at specified image locations and exposure control. The method for imaging a patient's body part also includes the step of registering the acquired data sets, the step of performing image processing on the acquired data sets and the step of recombining good data from the image processed data sets into a single image of the patient's body part. A non-transitory computer storage medium having instructions stored thereon, when executed, executes a method including the step of selecting an optical imaging device to image the patient's body part, the step of acquiring one or more data sets with the optical imaging device and the step of registering the acquired data sets. The method further includes the step of performing image processing on the acquired data sets and the step of recombining good data from the image processed data sets into a single image of the patient's body part.

Referring to FIG. 10 a system 1000 for imaging a patient's body part includes a server system 1004, an input system 1006, an output system 1008, a plurality of client systems 1010, 1014, 1016, 1018 and 1020, a communications network 1012 and a handheld or mobile device 1022. The system 1000 for imaging a patient's body part also includes additional components and may not include all of the components listed above. The server system 1004 includes one or more servers. One server 1004 may be the property of the distributor of any related software or non-transitory storage media. The input system 1006 may be utilized for entering input into the server system 1004, and includes any one of, some of, any combination of, or all of a keyboard system, a mouse system, a track ball system, a track pad system, a plurality of buttons on a handheld system, a mobile system, a scanner system, a wireless receiver, a microphone system, a connection to a sound system, and/or a connection and an interface system to a computer system, an intranet, and the Internet. The output system 1008 may be utilized for receiving output from the server system 1004, and includes any one of, some of, any combination of or all of a monitor system, a wireless transmitter, a handheld display system, a mobile display system, a printer system, a speaker system, a connection or an interface system to a sound system, an interface system to one or more peripheral devices and/or a connection and/or an interface system to a computer system, an intranet, and/or the Internet. The system 1000 for imaging a patient's body part may illustrate some of the variations of the manners of connecting to the server system 1004, which may be a website such as an information providing website. The server system 1004 may be directly connected and/or wirelessly connected to the plurality of client systems 1010, 1014, 1016, 1018 and 1020 and may be connected via the communications network 1012. Client systems 1020 may be connected to the server system 1004 via the client system 1018. The communications network 1012 may be any one of, or any combination of, one or more local area networks or LANs, wide area networks or WANs, wireless networks, telephone networks, the Internet and/or other networks. The communications network 1012 includes one or more wireless portals. The client systems 1010, 1014, 1016, 1018 and 1020 may be any system that an end user may utilize to access the server system 1004. The client systems 1010, 1014, 10110, 1018 and 1020 may be personal computers, workstations, tablet computers, laptop computers, game consoles, hand-held, network enabled audio/video players, mobile devices and/or any other network appliance. The client system 1020 may access the server system 1004 via the combination of the communications network 1012 and another system, which may be the client system 1018. The client system 1020 may be a handheld or mobile wireless device 1022, such as a mobile phone, a tablet computer or a handheld, network-enabled audio/music player, which may also be utilized for accessing network content. The client system 1020 may be a cell phone with an operating system or SMARTPHONE 1024 or a tablet computer with an operating system or IPAD 1026.

Referring to FIG. 11 a server system 1100 includes an output system 1130, an input system 1140, a memory system 1150, which may store an operating system 1151, a communications module 1152, a web browser module 1153, a web server application 1154 and a patient user body part imaging non-transitory storage media 1155. The server system 1100 may also include a processor system 1160, a communications interface 1170, a communications system 1175 and an input/output system 1180. The server system 1100 includes additional components and/or may not include all of the components listed above. The output system 1130 includes a monitor system, a handheld display system, a printer system, a speaker system, a connection or interface system to a sound system, an interface system to one or more peripheral devices and a connection and interface system to a computer system, an intranet, and/or the Internet. The input system 1140 includes a keyboard system, a mouse system, a track ball system, a track pad system, one or more buttons on a handheld system, a scanner system, a microphone system, a connection to a sound system and a connection and/or an interface system to a computer system, an intranet and the Internet (i.e., IrDA, USB). The memory system 1150 includes a long-term storage system, such as a hard drive, a short-term storage system, such as a random access memory or a removable storage system, such as a floppy drive or a removable drive and a flash memory. The memory system 1150 includes one or more machine-readable mediums that may store a variety of different types of information. The term machine-readable medium may be utilized to refer to any medium capable of carrying information that may be readable by a machine. A machine-readable medium may be a computer-readable medium such as a non-transitory storage media. The memory system 1150 may store one or more machine instructions for imaging a patient's body part. The operating system 1151 may control all software or non-transitory storage media 1155 and hardware of the server system 1100. The communications module 1152 may enable the server system 1004 to communicate on the communications network 1012. The web browser module 1153 may allow for browsing the Internet. The web server application 1154 may serve a plurality of web pages to client systems that request the web pages thereby facilitating browsing on the Internet. The processor system 1160 includes any one of, some of, any combination of, or all of multiple parallel processors, a single processor, a system of processors having one or more central processors and one or more specialized processors dedicated to specific tasks. The processor system 1160 may implement the machine instructions stored in the memory system 1150. The communication interface 1170 may allow the server system 1100 to interface with the network 1012. The output system 1130 may send communications to the communication interface 1170. The communications system 1175 communicatively links the output system 1130, the input system 1140, the memory system 1150, the processor system 1160 and/or the input/output system 1180 to each other. The communications system 1175 includes any one of, some of, any combination of, or all of one or more electrical cables, fiber optic cables, and/or sending signals through air or water (i.e., wireless communications). Sending signals through air and/or water includes systems for transmitting electromagnetic waves such as infrared and radio waves and/or systems for sending sound waves. The input/output system 1180 includes devices that have the dual function as the input and output devices. The input/output system 1180 includes one or more touch sensitive screens, which display an image and therefore may be an output device and accept input when a user presses the screens either his finger or a stylus. The touch sensitive screens may be sensitive to heat and/or pressure. One or more of the input/output devices may be sensitive to a voltage or a current produced by a stylus. The input/output system 1180 may be optional and may be utilized in addition to or in place of the output system 1130 and/or the input device 1140.

Referring to FIG. 12 apparatus 1201 for the acquisition and visualization of dental radiographic images which U.S. Pat. No. 7,505,558 teaches and which includes an X ray emitter device 1202, a radiographic sensor 1205 for acquiring a dental radiographic image, a processing unit 1206 for storing and visualizing the image on a monitor 1207 and a communication device 1208 for transmitting the image acquired by the radiographic sensor to the processing unit 1206. The communication device 1208 includes a first communication interface 1209 and second communication interface 1210 connected to the radiographic sensor 1205 and the processing unit 1206, respectively, for transmitting the commands to be given to the radiographic sensor 1205 and/or to receive the radiographic images acquired and transmitted by the radiographic sensor 1205 itself.

U.S. Pat. No. 7,457,656 teaches a medical image management system which allows any conventional Internet browser to function as a medical workstation. The system is used to convert medical images from a plurality of image formats to browser compatible format. The system is also used to manipulate digital medical images in such a way that multiple imaging modalities from multiple different vendors can be assembled into a database of Internet standard web pages without loss of diagnostic information. Medical imaging is important and widespread in the diagnosis of disease. In certain situations the particular manner in which the images are made available to physicians and their patients introduces obstacles to timely and accurate diagnoses of disease. These obstacles generally relate to the fact that each manufacturer of a medical imaging system uses different and proprietary formats to store the images in digital form. This means that images from a scanner manufactured by General Electric Corp. are stored in a different digital format compared to images from a scanner manufactured by Siemens Medical Systems. Images from different imaging modalities, such as ultrasound and magnetic resonance imaging (MRI), are stored in formats different from each other. Although it is typically possible to “export” the images from a proprietary workstation to an industry-standard format such as “Digital Imaging Communications in Medicine” (DICOM), Version 3.0, several limitations remain as discussed subsequently. In practice, viewing of medical images typically requires a different proprietary “workstation” for each manufacturer and for each modality. Currently, when a patient describes symptoms, the patient's primary physician often orders an imaging-based test to diagnose or assess disease. Days after the imaging procedure, the patient's primary physician receives a written report generated by a specialist physician who has interpreted the images. The specialist physician has not performed a clinical history and physical examination of the patient and often is not aware of the patient's other test results. Conversely, the patient's primary physician does not view the images directly but rather makes a treatment decision based entirely on written reports generated by one or more specialist physicians. Although this approach does allow for expert interpretation of the images by the specialist physician, several limitations are introduced for the primary physician and for the patient. The primary physician does not see the images unless he travels to another department and makes a request. It is often difficult to find the images for viewing because there typically is no formal procedure to accommodate requests to show the images to the primary physician. Until the written report is forwarded to the primary physician's office, it is often difficult to determine if the images have been interpreted and the report generated. Each proprietary workstation requires training in how to use the software to view the images. It is often difficult for the primary physician to find a technician who has been trained to view the images on the proprietary workstation. The workstation software is often “upgraded” requiring additional training. The primary physician has to walk to different departments to view images from the same patient but different modalities. Images from the same patient but different modalities cannot be viewed side-by-side, even using proprietary workstations. The primary physician cannot show the patient his images in the physician's office while explaining the diagnosis. The patient cannot transport his images to another physician's office for a second opinion.

U.S. Patent Publication No. 2004/0165791 teaches a dental image storage and retrieval apparatus which includes one or more client computing devices for displaying and processing dental images. The client devices are connected via a network to a dental image file server. The dental images are stored on the file server using a standardized naming format that allows the dentist to browse through the images without loading an intermediate database management program, making the dental images independent of whatever viewing or editing software program is chosen to actually view or edit the dental images. Dentists have long benefited from recorded images of their patient's teeth. For some time now, x-ray technology has provided a straightforward and cost-effective means for dentists to capture images of their patient's teeth. At a minimum, x-ray images are an important diagnostic tool, allowing the dentist to “see inside” the mouth, a single tooth and/or several teeth of the patient. X-ray dental images have a number of other benefits, in that pictures can then be stored in the patient's file for future reference, to allow the dentist to track problems in a patient's teeth over time. X-ray pictures can also be used to show a patient where defects may exist in the patient's teeth and to help the dentist explain suggested treatments to address those defects. Dental imaging has come a long way since the x-ray. Digital imaging of dental images is now becoming commonplace. Now dentists can choose to use a variety of imaging devices, such as intra-oral cameras, scanners, digital video and the like to capture images of their patient's teeth. The above-described benefits of x-rays have been improved with these modern imaging devices. One problem has arisen in conjunction with the increase in imaging technology. That problem is the need for equipment that will effectively manage those images. As the sheer volume of those images increase, enormous strain can be placed on the limited computer resources that are often present in dental offices. A variety of prior art solutions are available to dentists to assist in managing dental images. One well-known software package that can be used to manage dental images is Vipersoft that is essentially an index based database package (using C TREE database on Dentrix software package) that can be used to store, retrieve and otherwise manage a plurality of dental images. In a typical larger-scale dental office the Vipersoft data file will be stored on a central file server in the administration area of the dental office. This central file server will be connected to a plurality of client machines in the dental operating suites. The client machines in each of the suites will then be able to access the centrally stored data file.

One problem with the prior art is that, due to the nature of index databases, any time a dentist needs to access even a single image on the centrally stored data file from a client machine in a dental suite, the entire database is loaded from the central file server to the client machine. This can be an enormous strain on the otherwise limited computing resources of the dental office, straining the bandwidth of the local area network within the dental office and stressing the CPU and RAM of the local client machine. The proprietary nature of the database file name system can require a dentist to undergo an expensive and complicated file conversion should the dentist decide to switch to another dental image storage and retrieval system. A corruption of even a small part of the database index file could result in the loss of an entire collection of dental images.

U.S. Pat. No. 8,990,942 teaches instructions for a non-transitory computer-readable medium storing computer-executable application programming interface (API)-level intrusion detection.

The applicant hereby incorporates the above-referenced patents and patent publications into this patent application.

### SUMMARY OF INVENTION

The present invention is a computer-implemented method for integrating a non-supported dental imaging device into dental imaging software operating on a computer. The computer is coupled to a display which is capable of displaying dental x-rays and dental photographs. An originally supported dental imaging device has an API binary file with an original filename accessible to the computer.

In a first aspect of the present invention the computer-implemented method includes the step of creating a replacement alternate API binary file which contains equivalent functionality as the API binary file of the original supported dental imaging device.

In a second aspect of the present invention the computer-implemented method includes the step of placing the replacement alternate API binary file either onto or accessible to the computer. The replacement alternate API binary file has the same filename as does the original filename of the API binary file of the originally supported dental imaging device.

In a third aspect of the present invention the computer-implemented method includes the step of having the replacement alternate API binary file or files operated on by the dental imaging software by means of the computer. The dental imaging software is not aware the dental imaging software is not communicating with the originally supported dental imaging device.

In a fourth aspect of the present invention the computer-implemented method includes the step of having the replacement alternate API binary file deliver image data acquired by the non-supported imaging device to the dental imaging software.

In a fifth aspect of the present invention the computer-implemented method includes the step of renaming the original filename of the API binary file of the originally supported dental imaging device.

In a sixth aspect of the present invention the computer-implemented method includes the step of deleting the original filename or filenames of the API binary file or files of the originally supported dental imaging device.

In a seventh aspect of the present invention the computer includes a microprocessor and a memory coupled to the microprocessor.

In an eighth aspect of the present invention a non-transitory computer-readable medium storing computer-executable application programming interface (API) for use with the computer includes a set of instructions which allows integration of the non-supported dental imaging devices into a dental imaging software.

In a ninth aspect of the present invention the dental imaging software is a legacy dental imaging software.

In a tenth aspect of the present invention the dental imaging software is a proprietary dental imaging software.

In an eleventh aspect of the present invention a Markush group of non-supported dental imaging devices consists of 2D intraoral x-ray sensors, 3D intraoral x-ray sensors, 2D extraoral x-ray sensors, 3D extraoral x-ray sensors, dental camera, dental image data sources, dental imaging acquisition devices, dental images stored in a non-volatile memory such as either a hard disk drive or a flash drive, imaging plate scanners and other diverse dental image sources.

In a twelfth aspect of the present invention the computer operates to communicate, translate, forward, delete input received or sent to the dental imaging software by means of the replacement alternate API binary file or files in order to create expected requests and responses to and from the dental imaging software thereby allowing support for a specific previously unsupported dental imaging device by the dental imaging software while the dental imaging software is configured to support an originally supported imaging device.

In a thirteenth aspect of the present invention an alternate application programming interface (API) controls two or more connected dental imaging devices simultaneously.

Other aspects and many of the attendant advantages will be more readily appreciated as the same becomes better understood by reference to the following detailed description and considered in connection with the accompanying drawing in which like reference symbols designate like parts throughout the figures.

### DESCRIPTION OF THE DRAWINGS

FIG. 1 is a conceptual diagram of a networking system including a desktop computer, a laptop computer, a server, a server, a network, a server, a tablet device and a private network group according to U.S. Patent Publication No. 2013/0226993.

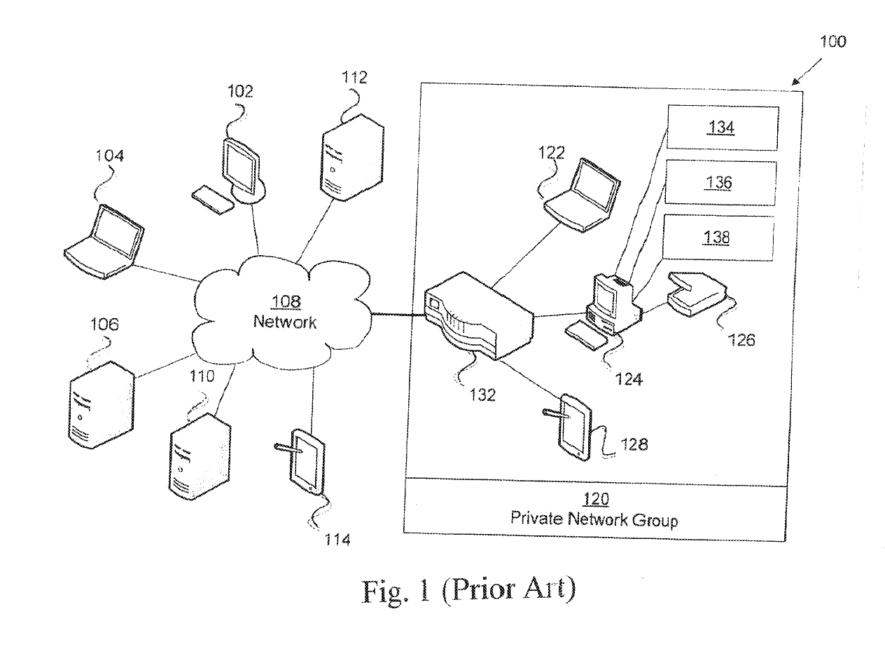

FIG. 2 is a conceptual diagram of a cloud-based server engine of the networking system of FIG. 1.

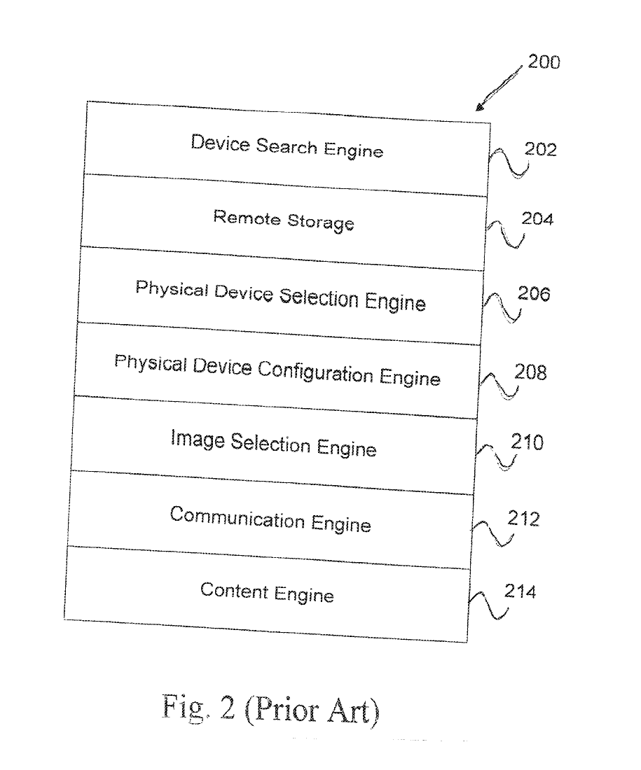

FIG. 3 is a conceptual diagram of a cloud-based client coupled to the networking system of FIG. 1.

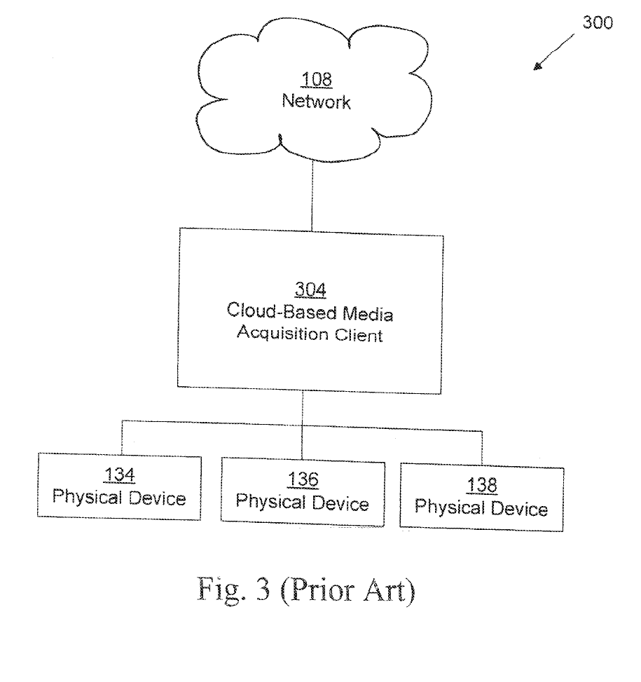

FIG. 4 is a schematic diagram of a networking system including a desktop computer, a laptop computer, a server, a server, a network, a server, a tablet device and a private network group according to U.S. Pat. No. 5,434,418.

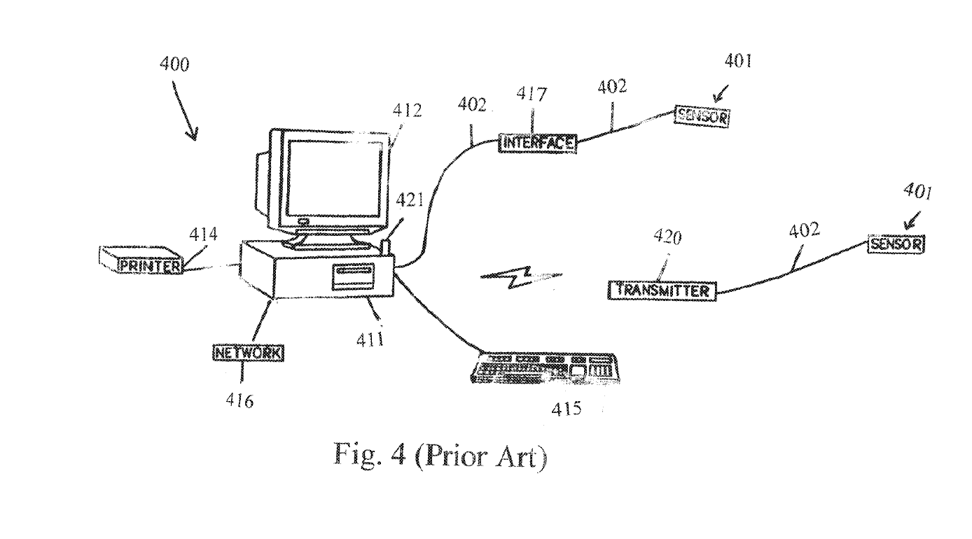

FIG. 5 is a schematic diagram of an x-ray diagnostics installation having a computer and a display according to U.S. Pat. No. 6,091,982.

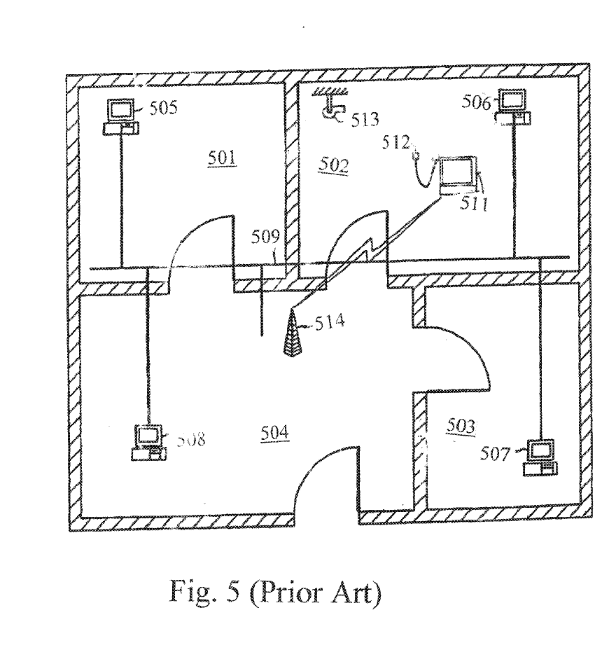

FIG. 6 is a schematic diagram of the computer and the display of the x-ray diagnostics installation of FIG. 5.

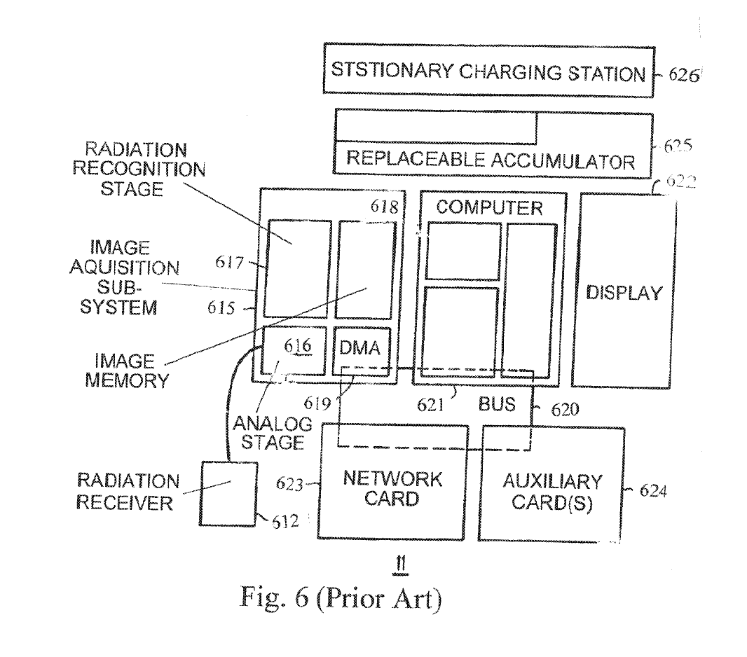

FIG. 7 is a schematic diagram of a mobile signal pick-up unit at a remote computer of the x-ray diagnostics installation of FIG. 5.

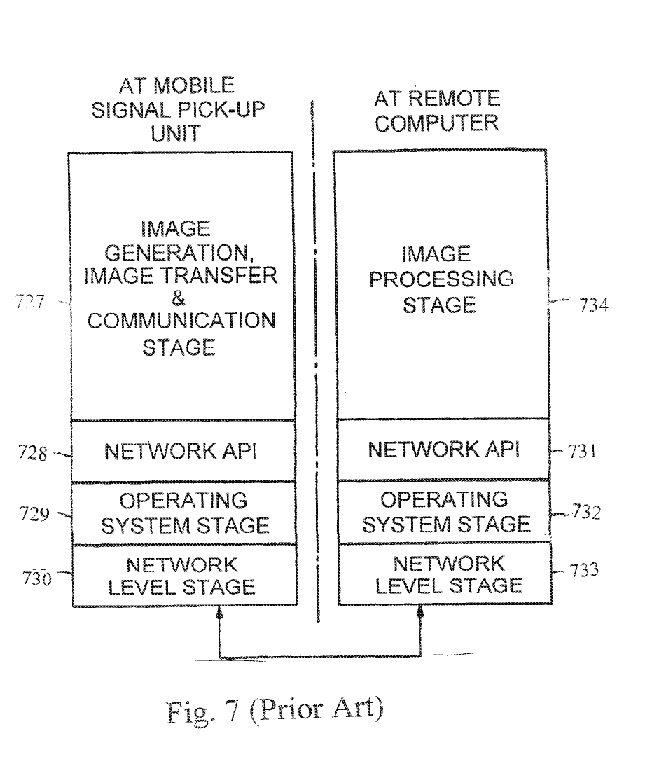

FIG. 8 is a schematic diagram of a universal image capture manager according to U.S. Patent Publication No. 2011/0304740.

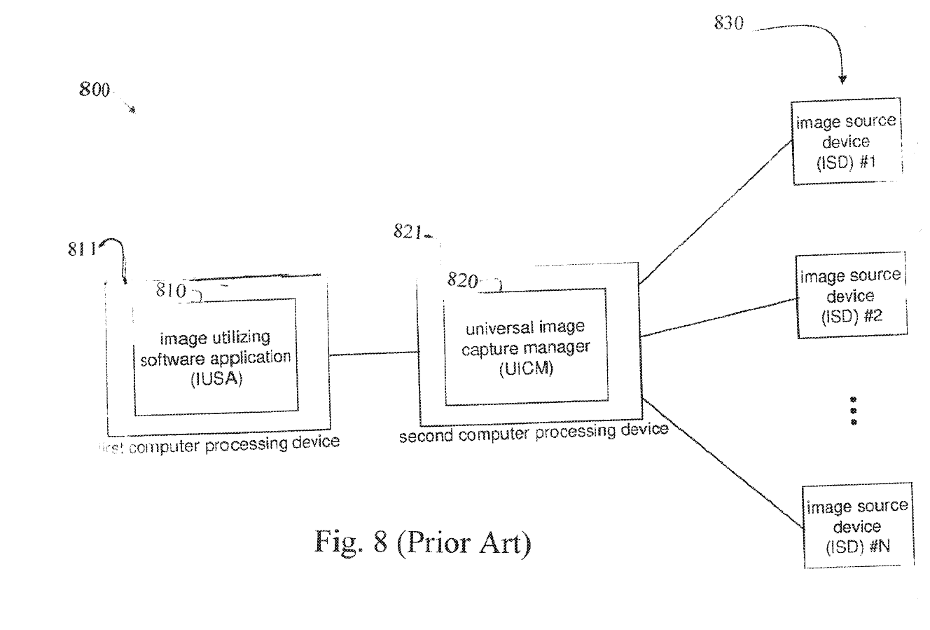

FIG. 9 is a schematic diagram of a software module architecture used in the universal image capture manager of FIG. 8.

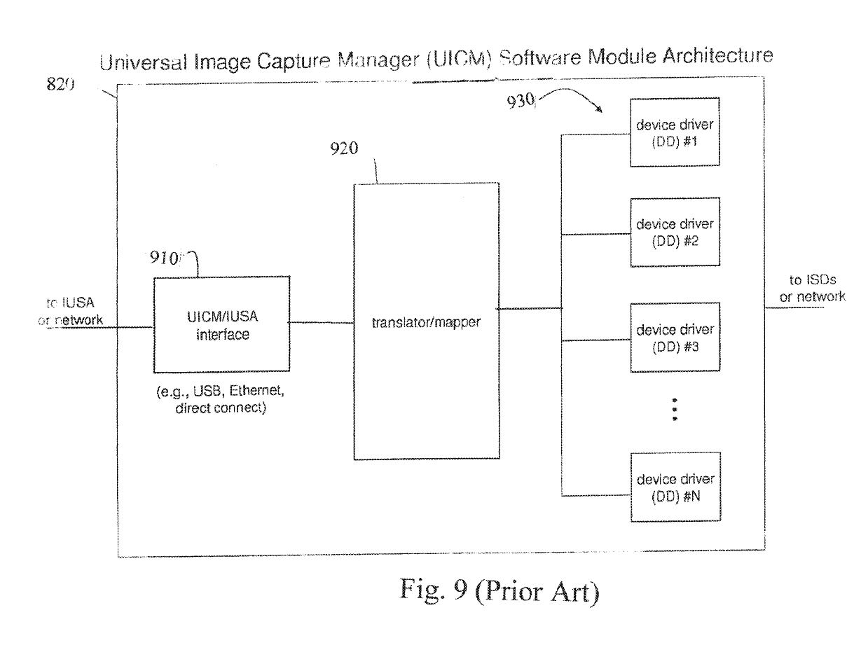

FIG. 10 is a schematic diagram of a system for imaging a patient's body part according to U.S. Patent Publication No. 2014/0350379.

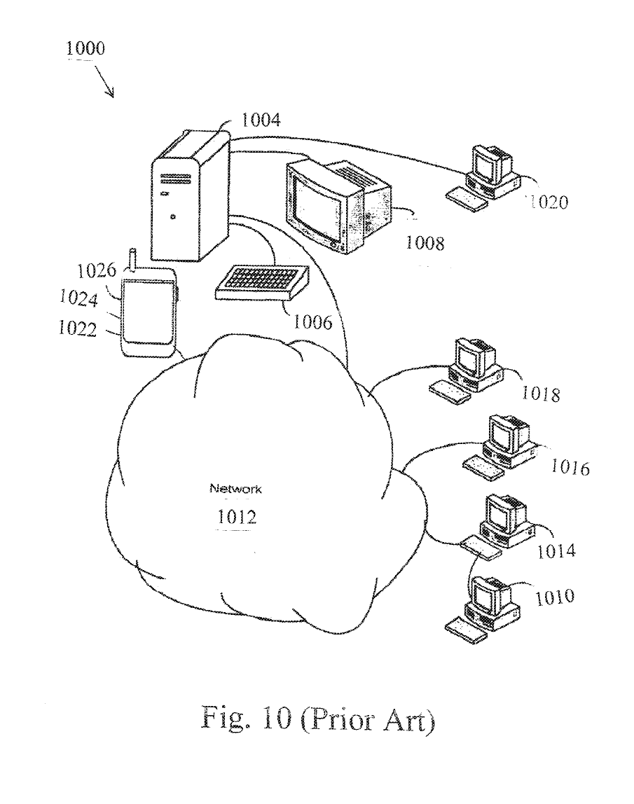

FIG. 11 is a schematic diagram of a server of the system for imaging a patient's body part of FIG. 10.

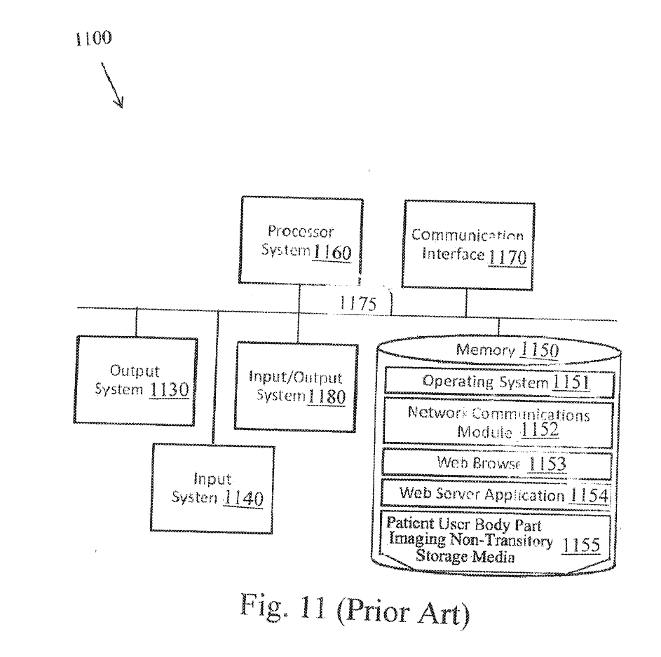

FIG. 12 is a schematic diagram of an apparatus 1201 for the acquisition and visualization of dental radiographic images according to U.S. Pat. No. 7,505,558

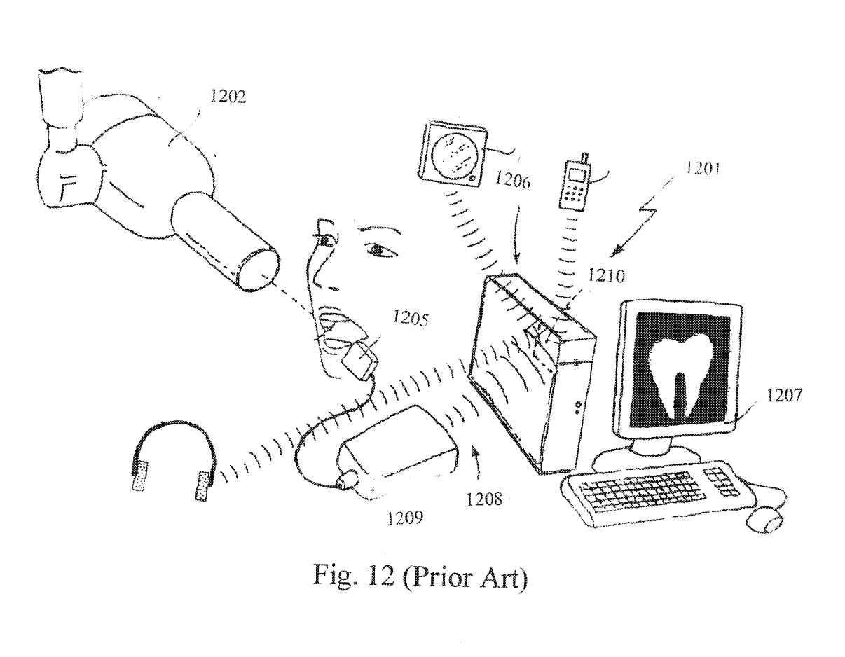

FIG. 13 is a schematic diagram of a dental office that uses proprietary or legacy dental imaging software and which is integrated to an originally supported imaging device but is not capable of integrating with an originally unsupported imaging device and is not using the claimed invention.

FIG. 14 is a schematic diagram of a dental office that uses proprietary or legacy dental imaging software and which is capable of integrating with an originally unsupported imaging device according to the present invention.

FIG. 15 is a schematic diagram of a flowchart of a method that integrates originally unsupported imaging devices into either legacy or proprietary dental imaging software according to the present invention.

### GLOSSARY OF TERMS

### Application Programming Interface (API)

An application programming interface (API) is a term used to describe a set of protocols, routines, functions and methods that specify the inputs, outputs, operations and underlying types of data and information for a specific software component and which component is meant to be integrated/used by another software component or application. API is a generalized term and includes interfacing via an executable, a control, or a library such as a dynamic link library. Functions, methods, parameters, messages are all API related references and are common types of methods used in APIs. The application programming interface (API) is a set of routines, protocols, and tools for building software.

### Binary API File

A Binary API File is defined as the physical API file that is physically located on the file system of a computing device and/or coupled to a computing device from another storage device.

### Renaming Binary API File

Renaming Binary API File is the physical act of renaming the Binary API file to another name on the file system or storage device.

### Replacement Binary API File

A Replacement Binary API File is a newly created physical file which is placed upon the computing device file system or coupled to it via another storage device and which physical file has identical name of the Binary API File. The Replacement API File has identical functions (APIs) exposed as the Binary API File. In other words, the Replacement API file is a Clone of the Binary API File from an “API” point of view.

### Selected/Current/Originally Supported/Native Dental Imaging Acquisition Device

A Selected/Current/Originally Supported/Native dental imaging acquisition device is referencing which specific proprietary dental imaging device or devices the dental imaging software is expected to acquire from; and which were originally programmed into that dental imaging software as a supported dental imaging device. This is a preference or setting in the dental imaging software that defines what acquisition device or devices can be used by the user of the imaging software. This may be a fixed setting (the dental imaging application software only supports a single or limited set of devices) or it may be from a menu or other means of user or system selection of supported non-standards based image acquisition devices. The selected or current device means the currently configured setting for a specific proprietary dental imaging device in the imaging software.

### Computer/Computing Device

A computer is a hardware microprocessor based computing device. Computer, computer device, computing device, computer hardware device terms are interchangeable. The computer is a physical hardware device and has a microprocessor, a RAM and a non-volatile memory. The computer has the ability to execute software. A display may be a monitor, computer screen or a display monitor. These terms are interchangeable. The computer is directly or indirectly coupled to the display.

### Legacy Imaging Software or Application

Legacy Imaging Application is an existing/older dental imaging software that is not updated regularly and/or is not updated to support specific imaging devices that have been released since the software has been in existence.

### Proprietary Imaging Software or Application

Proprietary Imaging Software or Application is a dental imaging software that via proprietary means supports dental imaging acquisition devices. The proprietary imaging application may support open standards as well such as Twain/Dicom communication and/or others but at a minimum at least one specific dental imaging device is supported via non-open standards via using an API to the specific imaging device.

### DESCRIPTION OF THE PREFERRED EMBODIMENT

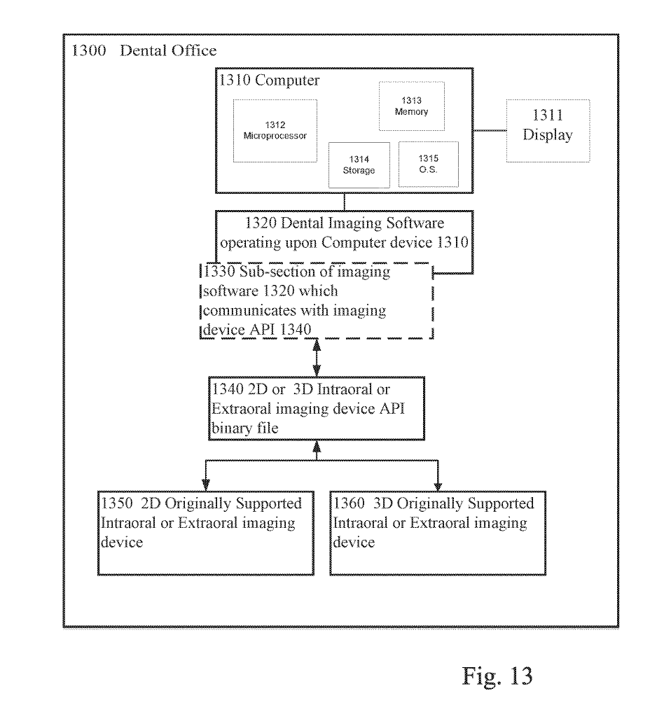

Referring to FIG. 13 a dental office 1300 includes a computer 1310 and a display 1311. The computer 1310 includes a microprocessor 1312, a memory 1313, such as a random access memory (RAM), and a non-volatile storage or memory 1314, such as either a hard disk or a flash memory, for storing software or data. The computer 1310 may be coupled either directly or indirectly to the display 1311. The display 1311 is capable of displaying dental images including dental x-rays and dental photographs. The computer 1310 has an operating system 1315 which may be either a Windows based operating system or a Mac OS X based operating system or another compatible operating system. The computer 1310 may also be a mobile computer, such as an iPad, an Android based tablet, a Microsoft Surface based tablet, a phone, or any other proprietary device with an adequate microprocessor, an operating system and a display which is capable of displaying dental images including dental x-rays and dental photographs.

Still referring to FIG. 13 the dental office 1300 also includes dental imaging software 1320 having a sub-section 1330 which integrates and acquires images from a specific supported or proprietary imaging device using the API binary file 1340 of the specifically supported proprietary imaging device. The dental imaging software 1320 is either legacy dental imaging software or proprietary dental imaging software. The first imaging device 1350 is a specifically originally supported native imaging device. The second imaging device 1360 is also a specifically supported native imaging device. The first imaging device 1350 may be either a 2D intraoral or a 2D extraoral dental imaging device. The second imaging device 1360 may be either a 3D intraoral or a 3D extraoral dental imaging device. The group of supported dental imaging devices may consist of 2D intraoral x-ray sensors, 3D intraoral x-ray sensors, 2D extraoral x-ray sensors, 3D extraoral x-ray sensors, dental camera, dental image data sources, dental imaging acquisition devices, dental images stored in a non-volatile memory such as either a hard disk drive or a flash drive, imaging plate scanner sensors and any other diverse dental image sources.

Referring still further to FIG. 13 the dental office 1300 does not use the claimed invention. The computer 1310 operates imaging software 1320. The dental imaging software 1320 may be either running locally on the computer 1310 or displaying the results of software operating upon a remote server, such as either web-based dental imaging software or cloud-based dental imaging software. The imaging software 1320 may be either directly controlling or indirectly controlling the first imaging device 1350 using sub-section 1330 of the dental imaging software 1320. The dental imaging software 1320 may also be either directly or indirectly controlling the second imaging device 1360 using sub-section 1330 of the dental imaging software 1320. The sub-section 1330 communicates with the API binary file 1340 which in turn communicates with at least one of the first and second imaging devices 1350 and 1360 to direct imaging or receive images. The API binary file 1340 is stored in either the non-volatile storage 1314, or the memory 1313 on the computer 1310 or in another either non-volatile storage or memory either coupled to the computer 1310 or accessible by computer 1310. The Imaging software 1320 communicates to the sub-section 1330 for the purpose of controlling the actions of at least one of the first and second imaging devices 1350 or 1360 using the API binary file 1340 thereof. The sub-section 1330 receives communication or status from the specific imaging device by means of its API binary file 1340 which communicates directly or indirectly with the device driver of one of the first and second imaging devices 1350 and 1360. The communications between the imaging software 1320 and sub-section 1330 and to binary imaging device API binary file 1340 are proprietary in nature. The API binary files are not universal for imaging devices and no two imaging devices typically have the same functions, parameters, or overall operation in their API binary file for that specific imaging device. Dental imaging software 1320 commands the computer 1310 to initiate and/or receive image or image data from either the first imaging device 1350 or the second imaging device 1360 by means of communication through sub-section 1330 and its API binary file 1340. After either an image or image data has been enacted by API binary file 1340 it is made available to the dental imaging software 1320 by means of the sub- section 1330 or other means for any additional processing, storage and ultimately display upon computer 1310.

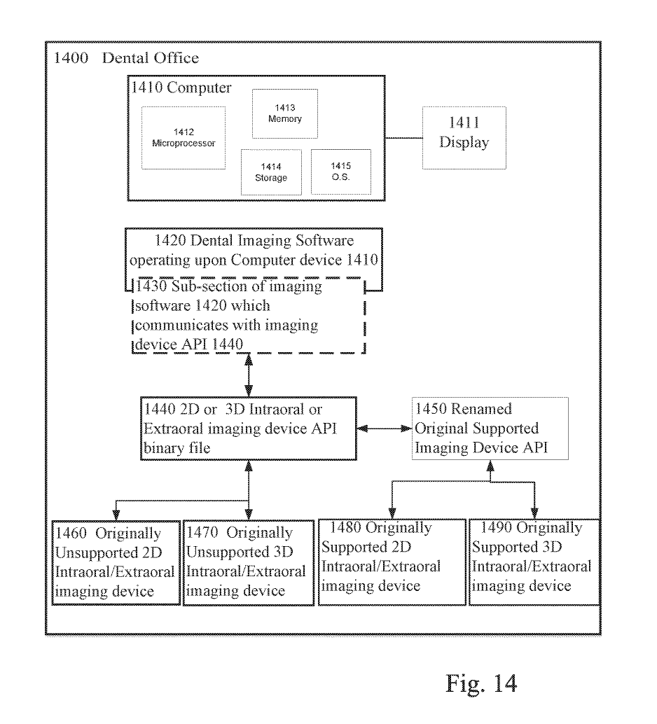

Referring to FIG. 14 a dental office 1400 includes a computer 1410 and a display 1411. The computer 1410 includes a microprocessor 1412, a memory 1413, such as a random access memory (RAM), and a non-volatile storage or memory 1414, such as either a hard disk or a flash memory, for storing software or data. The computer 1410 may be coupled either directly or indirectly to the display 1411. The display 1411 is capable of displaying dental images including dental x-rays and dental photographs. The computer 1410 has an operating system 1415 which may be either a Windows based operating system or a Mac OS X based operating system, or another compatible operating system.

Referring still to FIG. 14 the dental office 1400 uses the claimed invention. The computer 1410 operates imaging software 1420. The computer 1410 may also be a mobile computer, such as an iPad, an Android based tablet, a Microsoft Surface based tablet, a phone or any other proprietary device with an adequate microprocessor, operating system and display capability. The dental office 1400 also includes imaging software 1420 having a sub-section 1430 which integrates and acquires images from a specific or proprietary imaging device using the API binary file 1440 of the specific or proprietary imaging device. The first imaging device 1460 is an originally unsupported 2D imaging device. The second imaging device 1470 is also an originally unsupported 3D imaging device. The first imaging device 1460 may be a 2D intraoral or extraoral dental imaging device. The second imaging device 1470 may be a 3D intraoral or extraoral dental imaging device. Originally supported dental imaging devices may consist of 2D intraoral x-ray sensors, 3D intraoral x-ray sensors, 2D extraoral x-ray sensors, 3D extraoral x-ray sensors, dental camera, dental image data sources, dental imaging acquisition devices, dental images stored in a non-volatile memory such as either a hard disk drive or a flash drive, imaging plate scanner sensors, PSP devices and any other diverse dental image sources. Originally non-supported dental imaging devices that become supported using the claimed invention may consist of 2D intraoral x-ray sensors, 3D intraoral x-ray sensors, 2D extraoral x-ray sensors, 3D extraoral x-ray sensors, dental camera, dental image data sources, dental imaging acquisition devices, dental images stored in a non-volatile memory such as either a hard disk drive or a flash drive, imaging plate scanner sensors, PSP devices and any other diverse dental image sources.

Referring still further to FIG. 14 the imaging software 1420 may be either running locally on the computer 1410 or displaying the results of software operating upon a remote server, such as web/cloud based imaging software. The imaging software 1420 is communicating and/or controlling an originally/natively supported 2D intraoral or extraoral imaging device 1480 and/or an originally supported 3D intraoral or extraoral imaging device 1490. Sub-section 1430 of the imaging software includes integration to a specific proprietary imaging device API binary file 1440. The original specific imaging device API binary file 1440 that sub-section 1430 communicated with has been renamed to a different filename on computer 1410 or on another device accessible to computer 1410. The renamed original API binary 1450 is accessible to replacement API binary file 1440. The filename of replacement binary API file 1440 is named the same as the original specific proprietary imaging device API binary filename and contains identical or near-identical functions as original API binary which are called by the decoupled imaging software to support the specific imaging device natively. The specific imaging device is a previously supported 2D intraoral or extraoral dental imaging device 1480 or 1490 3D imaging device and/or the imaging device is a previously unsupported 2D imaging device 1460 or 3D intraoral or extraoral dental imaging device 1470.

Referring yet still further to FIG. 14 the subsection 1430 of the imaging software 1420 includes a replacement API binary file 1440 and shows the dental imaging software sub-section 1430 communicating with the replacement API binary and controlling either the original natively supported imaging devices 1480 and/or 1490 or the non-natively supported imaging devices 1460 and/or 1470. The imaging software is unaware that it is not communicating with the original natively supported imaging device API binary as the function/parameters called and values returned are identical to the original natively supported API binary file in the replacement API binary file 1440. When the imaging software sub-section 1430 communicates with replacement API binary 1440, the said API binary file 1440 communicates with the renamed original API binary file 1450 and relays the same functions and parameters as were communicated to it by means of the imaging software 1420 and its sub-section 1430; and which allows the original natively supported imaging devices 1480 and 1490 to continue to be supported in imaging software 1420. Replaced binary API file 1440 also communicates with non-supported imaging device 1460 and 1470 API/devices. Replacement API 1440 translates, forwards, adds and deletes functions or parameters received from the imaging software to be compatible with the previously non-supported imaging device 1460 or 1470 and their API. Replaced binary API file 1440 also translates or converts imaging device 1460 or 1470's API return codes, messages, or image data to be compatible with what sub-section 1430 of imaging software 1420 expects for functions, return values, and messages from the API binary file 1440. The imaging software subsection 1430 calls the same functions and/or parameters and/or methods for the originally natively supported device via the replacement API binary 1440 which has the same functions as the original renamed API binary 1450. The imaging software is not aware of any changes or that it is not communicating with the natively supported imaging devices via the original API binary file or files. The existing natively supported imaging devices 1480 and 1490 continue to operate and non-natively supported devices 1460 and 1470 can now operate within the decoupled imaging software. This is one hundred per cent (100%) transparent to the imaging software so that no changes are required to the legacy or proprietary imaging software application.

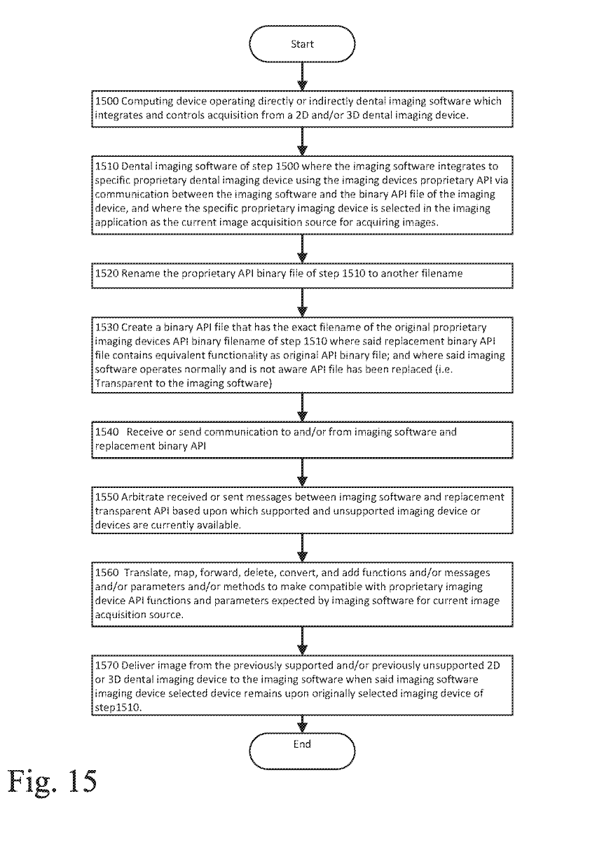

Referring to FIG. 15 and referencing FIG. 14 a computer-implemented method for integrating a non-supported dental imaging device into dental imaging software operates on the computer 1410 coupled to the display 1411 which is capable of displaying dental x-rays and dental photographs. An originally supported dental imaging device has either an API binary file or API binary files with either an original filename or filenames, respectively, either directly or indirectly accessible to the computer 1410. The computer-implemented method 1500 includes the steps of operating a legacy or proprietary dental imaging software application which controls acquisition from a 2D or 3D imaging device upon a computing device. In step 1510 the proprietary or legacy imaging software has been programmed to support specific 2D and/or 3D imaging devices using proprietary API's, and where the imaging software is configured to acquire images from one or more of the supported imaging devices. In step 1520 the original binary API file or files for an originally supported device has been renamed to another filename. In step 1530 a replacement API file with the same filename as the original API filename has been created and placed onto or accessible to the computing device. In step 1530 the replacement API file is enacted upon by the imaging software to acquire images and is not aware it is not communicating with the original supported device API. In step 1540 communication is received or initiated between the imaging software and the replacement binary API. In step 1550 any messages sent or received from devices or the legacy application are arbitrated to the proper proprietary API/device. In step 1560 any communicated between the API and the imaging software are translated, converted, etc. . . . transparently to the imaging application software. In step 1570 the image or image data is delivered from the previously supported or un-supported device transparently in that the imaging software does not know it is not receiving images or communication from the originally supported device and device API. Thereby allowing support for a specific previously unsupported dental imaging device by the dental imaging software while the dental imaging software is configured to support an originally supported imaging device. The computer-implemented method may include either the step of renaming the original filename or filenames of either the API binary file or the API binary files of the originally supported dental imaging device on the computer 1410 or the step of deleting either the original filename or the original filenames of either the API binary file or the API binary files of the originally supported dental imaging device on the computer 1410. An alternate application programming interface (API) may control two or more connected dental imaging devices simultaneously. The computer-implemented method includes a non-transitory computer-readable medium storing computer-executable application programming interface (API) for use with the computer. The non-transitory computer-readable medium includes a set of instructions which allows integration of the non-supported imaging devices into dental imaging software.

From the foregoing it can be seen that integration of non-supported dental imaging devices into legacy and proprietary dental imaging software has been described. It should be noted that the sketches are not drawn to scale and that distances of and between the figures are not to be considered significant.

Accordingly it is intended that the foregoing disclosure and showing made in the drawing shall be considered only as an illustration of the principle of the present invention.

What is claimed is:

1. A computer-implemented method for integrating a non-supported dental imaging device into dental imaging software operating on a computer coupled to a display being capable of displaying dental x-rays and dental photographs wherein an originally supported dental imaging device has an API binary file or files with an original filename or filenames, respectively, either directly or indirectly accessible to the computer, said computer-implemented method comprises the steps of:
a. creating a replacement alternate API binary file or files which contain equivalent functionality as the API binary file or files of the original supported dental imaging device;
b. placing said replacement alternate API binary file or files either onto or accessible to the computer wherein said replacement alternate API binary file or files have the same filename or filenames as do the original filename or filenames of the API binary file or files of the originally supported dental imaging device;
c. having said replacement alternate API binary file or files operated on by the dental imaging software by means of the computer wherein the dental imaging software is not aware the dental imaging software is not communicating with the originally supported dental imaging device; and
d. having said replacement alternate API binary file or files deliver image data acquired by the non-supported imaging device to the dental imaging software.

2. A computer-implemented method for integrating a non-supported dental imaging device into dental imaging software operating on a computer according to claim 1 wherein said computer-implemented method includes the step of renaming the original filename or filenames of the API binary file or files of the originally supported dental imaging device on the computer.

3. A computer-implemented method for integrating a non-supported dental imaging device into dental imaging software operating on a computer according to claim 1 wherein said computer-implemented method includes the step of deleting the original filename or filenames of the API binary file or files of the originally supported dental imaging device on the computer.

4. A computer-implemented method for integrating a non-supported dental imaging device with dental imaging software operating on a computer according to claim 1 wherein the computer includes a hardware-based microprocessor and a memory coupled to the microprocessor.

5. A computer-implemented method for integrating a non-supported dental imaging device with dental imaging software operating on a computer according to claim 1 includes a non-transitory computer-readable medium storing computer-executable application programming interface (API) for use with the computer that includes a set of instructions which allows integration of the non-supported dental imaging devices into a dental imaging software.

6. A computer-implemented method for integrating a non-supported dental imaging device with dental imaging software operating on a computer according to claim 1 wherein the dental imaging software is a legacy dental imaging software application.

7. A computer-implemented method for integrating a non-supported dental imaging device with dental imaging software operating on a computer according to claim 1 wherein the dental imaging software application is a proprietary dental imaging software application.

8. A computer-implemented method for integrating a non-supported dental imaging device with dental imaging software operating on a computer according to claim 1 wherein a Markush group of non-supported dental imaging devices consists of 2D intraoral x-ray sensors, 3D intraoral x-ray sensors, 2D extraoral x-ray sensors, 3D extraoral x-ray sensors, dental camera, dental image data sources, dental imaging acquisition devices, dental images stored in a non-volatile memory such as either a hard disk drive or a flash drive, imaging plate scanner sensors and other diverse dental image sources.

9. A computer-implemented method for integrating a non-supported dental imaging device with dental imaging software operating on a computer according to claim 1 wherein a computer operates to communicate, translate, forward, delete input received or sent to the dental imaging software by means of said replacement alternate API binary file or files in order to create expected requests and responses to and from the dental imaging software thereby allowing support for a specific previously unsupported dental imaging device by the dental imaging software while the dental imaging software is configured to support an originally supported imaging device.

10. A computer-implemented method for integrating a non-supported dental imaging device with dental imaging software operating on a computer according to claim 1 wherein an alternate application programming interface (API) controls two or more connected dental imaging devices simultaneously.

11. A computer-implemented method for integrating a non-supported dental imaging device into dental imaging software operating on a computer coupled to a display being capable of displaying dental x-rays and dental photographs wherein an originally supported dental imaging device has an API binary file with an original filename accessible to the computer, said computer-implemented method comprises the steps of:
a. creating a replacement alternate API binary file which contains equivalent functionality as the API binary file of the original supported dental imaging device;
b. placing said replacement alternate API binary file either onto or accessible to the computer wherein said replacement alternate API binary file has the same filename as does the original filename of the API binary file of the originally supported dental imaging device;
c. having said replacement alternate API binary file operated on by the dental imaging software by means of the computer wherein the dental imaging software is not aware the dental imaging software is not communicating with the originally supported dental imaging device; and
d. having said replacement alternate API binary file deliver image data acquired by the non-supported imaging device to the dental imaging software.

12. A computer-implemented method for integrating a non-supported dental imaging device into dental imaging software operating on a computer according to claim 11 wherein said computer-implemented method includes the step of renaming the original filename of the API binary file of the originally supported dental imaging device on the computer.

13. A computer-implemented method for integrating a non-supported dental imaging device into dental imaging software operating on a computer according to claim 11 wherein said computer-implemented method includes the step of deleting the original filename of the API binary file of the originally supported dental imaging device on the computer.
